(19) United States
(12) Patent Application Publication
(10) Pub. No.: US 2007/0161628 A1
(43) Pub. Date: Jul. 12, 2007

(54) Phosphodiesterase inhibitors

(75) Inventor: Philippe Bernard, Orleans (FR)

Correspondence Address: OLIFF & BERRIDGE, PLC, P.O. BOX 19928, ALEXANDRIA, VA, 22320, US

(73) Assignee: GREENPHARMA, SAINT BEAUZIRE (FR)

(21) Appl. No.: 11/601,778

(22) Filed: Nov. 20, 2006

Related U.S. Application Data
(63) Continuation-in-part of application No. PCT/FR05/01260, filed on May 19, 2005.

(30) Foreign Application Priority Data
May 19, 2004 (FR) 04.0551

(51) Int. Cl. A61K 31/5513; C07D 243/10; C07D 403/02

(52) U.S. Cl. 514/221; 540/500; 540/567

(57) ABSTRACT

The invention relates especially to novel stereospecific derivatives of 2,3-benzodiazepine type as inhibitors of phosphodiesterases, especially 2 and 4, and uses thereof in the therapeutic field, most particularly for preventing and treating pathologies involving a central and/or peripheral disorder. The compounds of the invention more particularly correspond to the general formulae (I) and (II):

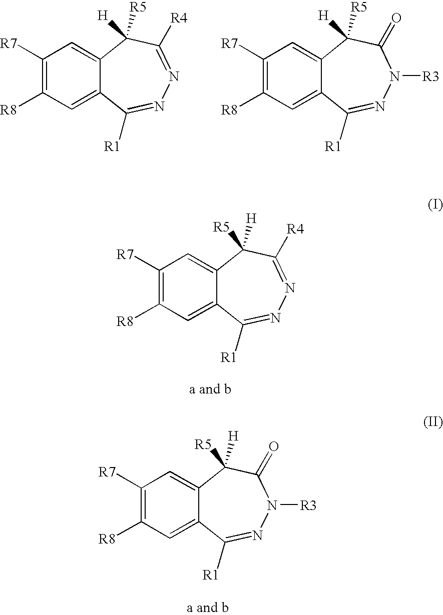

The invention relates to the use of molecules with inhibitory activities on phosphodiesterases 2 and 4, for neurotrophic and neuroprotective properties, which can thus be used as human or veterinary medicaments.

Under physiological conditions, neurites (dendrites and axons) allow the neurons to make a large number of connections with neighbouring neurons. These neurons can transmit messages across synapses by means of messengers or neurotransmitters such as catecholamines, amino acids or peptides. When these connections between neurons reduce, following cell death or age-related degeneration, diseases, disorders or traumas, the mental capacities of the individual may be seriously impaired.

Carbon monoxide, which is especially produced by an enzyme, haem oxygenase, functions as a neurotransmitter and is capable of inducing, after diffusion into a cell, the production of a second cell messenger: cyclic guanosine monophosphate (cGMP). This induction of cGMP is performed by means of a carbon monoxide-dependent guanylate cyclase. Moreover, cGMP, just like cAMP, is degraded by a family of enzymes, the phosphodiesterases (PDE), which are divided into at least 11 groups. By slowing down the degradation of cGMP and cAMP, PDE inhibitors increase or maintain the level of cGMP and cAMP in cells and prolong their biological effects.

It is established that increasing the intracellular levels of cGMP results in a modification of numerous cellular activities, and especially of the synthesis and release of several endogenous neurotrophic factors (neurotrophin and pleiotrophin) and also of other neuronal factors, which may induce, promote or modify a wide variety of cell functions, especially cell growth and communication.

Neurotrophic factors are molecules that exert a very wide variety of biological effects and stimulate the development and differentiation of neurons and maintenance of cell integrity, and are necessary for the survival and development of neurons. More particularly, neurotrophic factors make it possible to prevent neuronal death and to stimulate the growth of neurites and also to reduce membrane potentials, making the neuron more receptive to cell signals. Growth factors may also change the long-term potentiation of neurons, inducing an increase in neuronal plasticity and allowing an increase in cognitive and mental faculties.

Neuronal functions are impaired in certain central or peripheral conditions or diseases. Among these conditions or diseases usually resulting from excessive neuronal death, mention may be made especially, in a non-limiting manner of: ageing, Alzheimer's disease, Parkinson's disease, amyotrophic lateral sclerosis, multiple sclerosis, Huntington's disease, strokes, peripheral neuropathies, retinopathies (especially pigmentary retinitis), prion diseases (especially spongiform encephalopathies such as Creutzfeldt-Jakob disease), traumas (spinal column accidents, compression of the optic nerve following glaucoma, etc.) or neuronal disorders caused by the action of chemical products, and also disorders associated with these conditions or diseases that may be disorders secondary to the primary pathology. In many cited cases, it is usually the progressive death of motor neurons that is the cause of the disorders observed, and conventional treatments call upon the administration of anti-inflammatory agents to avoid the onset of secondary disorders.

One of the means for preventing such impairments and/or for re-establishing damaged neuronal function is to regenerate neurites between the various nerve cells, for example, by increasing the local concentrations of one or more growth factors. Treatments involving small molecules capable of increasing the synthesis and/or secretion of growth factors and which preferentially act via the oral or injection route will be preferred to those using natural growth factors, which are large, orally inactive molecules incapable of penetrating into the central nervous system. By inducing the secretion and/or synthesis of growth factors, these small molecules are also capable of changing the long-term potentiation of neurons, inducing, especially in the hippocampus, an increase in neuronal plasticity, which will have the consequence of increasing the cognitive and mental faculties.

Moreover, type 2 and 4 phosphodiesterase (PDE2 and PDE4) inhibitors are capable, by increasing the intracellular concentration of cAMP, of exerting a cytoprotective effect and of especially increasing the survival of dopaminergic neurons (Pérez-Torres, S. et al. J. Chem. Neuroanatomy, 2000, 20, 349-374). It has also been reported that cAMP is involved in the transduction of many neurotransmitters and hormones and can thus especially modulate the effect of growth factors.

By slowing down the degradation of cAMP, a PDE4 or PDE2 inhibitor can consequently produce a neurological and/or neuroprotective effect.

It is moreover known that PDE4 inhibitors represent potential treatments for many central or peripheral diseases, especially autoimmune and inflammatory diseases.

The field of application of PDE4 inhibitors especially covers the treatment and prevention of inflammation and of lack of bronchial relaxation, and more particularly of asthma and chronic obstructive bronchopathies, but also other conditions, for instance rhinitis, acute respiratory distress syndrome, allergies, dermatitis, psoriasis, rheumatoid arthritis, multiple sclerosis, dyskinesia, glomerulonephritis, osteoarthritis, cancer, septic shock, AIDS, Crohn's disease, osteoporosis or obesity.

IPDE4s are also endowed with central effects that are particularly advantageous for treating depression, anxiety, schizophrenia, bipolar disorder, attention deficits, fibromyalgia, Parkinson's and Alzheimer's diseases, amyotrophic sclerosis, multiple sclerosis, Lewy body dementia and other psychiatric disorders.

International patent applications WO 02/088096 and WO 02/098865 describe racemic tofisopam derivatives as phosphodiesterase 4 inhibitors. This patent application does not take into account pure stereochemical compounds.

International patent application WO 00/24400 describes the mode of preparation and the use of (R)-tofisopam, one of the two optically pure compounds of tofisopam, for treating anxiety. This patent application does not deal either with phosphodiesterases or with other tofisopam isomers. It demonstrates better efficacy of the stereochemically pure compound while reducing the side effects that may be induced by the racemic mixture.

International patent application WO 02/45749 describes phosphodiesterase 4 inhibitors as agents capable of reversing the inhibition of the neuronal regeneration of the central and peripheral nervous systems in mammals. PDE2 inhibitors are not concerned.

Patent application U.S. Pat. No. 6,638,928 of 28 Oct. 2003 describes derivatives of 2,3-benzodiazepine type for treating diseases such as Irritable Bowel Syndrome and non-ulcer dyspepsia. This patent mentions neither PDE2 and PDE4 inhibitors nor the treatment of diseases of the central nervous system.

The Applicant has now demonstrated that the compounds according to the invention are capable of stimulating neurotrophic activity and/or of maintaining a cytoprotective/neuroprotective effect by virtue of the inhibitory properties on phosphodiesterases, especially PDE2 and/or PDE4.

One subject of the invention is novel molecules with inhibitory properties on type 2 and 4 phosphodiesterases and the use thereof, but also the use of known compounds for the preparation of compounds that inhibit type 2 and 4 phosphodiesterases. This use may be performed with combinations of compounds having one or other of the properties, or alternatively a compound combining both properties.

It is thus possible to prepare a pharmaceutical composition incorporating all compounds known for inhibiting type 2 phosphodiesterase with compounds known for inhibiting phosphodiesterase 4.

Overcoming the effect on the phosphodiesterase of subtype 4D will be a plus. Among the known compounds that may be used, mention may be made, in a non-limiting manner, of papaverine, amentoflavone and derivatives thereof or any other natural or synthetic molecules capable of more or less selectively inhibiting phosphodiesterases 2 and 4, or synthetic molecules, for instance tofisopam or tofisopam analogues, already described and synthesized, for example, in the abovementioned patents. Novel compounds have also been synthesized.

In this context, a subject of the invention is also compounds corresponding to the general formulae (I) and (II)

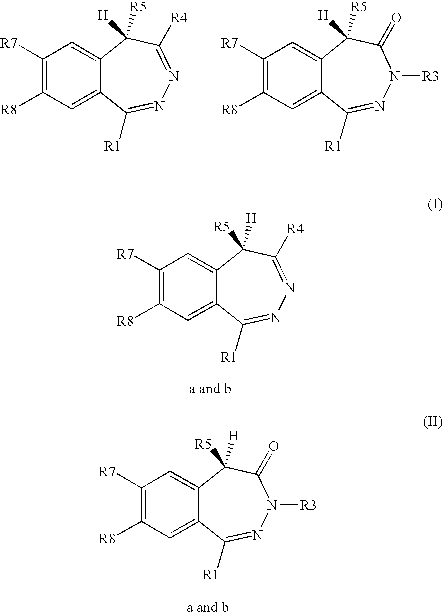

in which:
R₁ and R₃, independently of each other, are chosen from a hydrogen atom, a group (C₁-C₆) alkyl, (C₃-C₆) cycloalkyl, (C₆-C₁₈) aryl, (C₆-C₁₈)aryl(C₁-C₄)alkyl, (C₁-C₆)alkyl(C₆-C₁₈)aryl, (C₅-C₁₈) heteroaryl comprising 1 to 3 heteroatoms, or a group OR₂, SR₂ or NR₂R_{3′} in which (i) R₂ and R_{3′}, independently of each other, are chosen from a hydrogen atom, and a group(C₁-C₆) alkyl, (C₃-C₆) cycloalkyl, (C₆-C₁₂) aryl, (C₅-C₁₂) heteroaryl comprising 1 to 3 heteroatoms or (ii) R₂ and R_{3′} together form a linear or branched hydrocarbon-based radical containing from 2 to 6 carbon atoms, optionally comprising one or more double bonds and/or optionally interrupted with an oxygen, sulfur or nitrogen atom,
R₄ is chosen from a halogen atom, a (C₁-C₇) alkyl, (C₂-C₇) alkenyl, (C₂-C₇) alkynyl, or phenyl group or a group (C═O)R₂, OR₂, SR₂ or NR₂R_{3′} in which R₂ and R_{3′} are as defined above,
R₅ is chosen from (C₁-C₆) alkyl, (C₂-C₆) alkenyl, (C₃-C₆) cycloalkyl and (C₂-C₆) alkynyl groups,
R₇ and R₈, independently of each other, are chosen from a hydrogen atom, a (C₁-C₆) alkyl group or a group OR₂, SR₂ or NR₂R_{3′} in which R₂ and R_{3′} are as defined above,

the alkyl, cycloalkyl, aryl, heteroaryl, alkenyl and alkynyl groups and the hydrocarbon-based chain defined above being optionally substituted with one or more identical or different substituents preferably chosen from a halogen atom, an OH, ═O, NO₂, NH₂, CN, COOH or CF₃ group, a (C₁-C₆) alkoxy group and a group NHCOR₂ or CONR₂R_{3′}, in which R₂ and R₃ are as defined above.

The compounds of the invention may be in the form of salts, especially of basic or acidic addition salts, which are preferably compatible with pharmaceutical use. Among the pharmaceutically acceptable acids that may be mentioned, in a non-limiting manner, are hydrochloric acid, hydrobromic acid, sulfuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, methane sulfonic acid, ethane sulfonic acid, camphoric acid, etc. Among the pharmaceutically acceptable bases that may be mentioned, in a non-limiting manner, are sodium hydroxide, potassium hydroxide, triethylamine, tert-butylamine, etc.

The invention also concerns the compounds mentioned above, which are novel or known in the form of a mixture of enantiomers or of racemic isomers or enriched in an isomer and/or in optically pure form, for example in R or S form.

The invention more particularly concerns S-tofisopam, or (5S)-1-(3,4-dimethoxyphenyl)-5-ethyl-7,8-dimethoxy-4-methyl-5H-2,3-benzodiazepine.

According to the invention, at least one of the atoms of the molecules described may be replaced with an isotope (atom having the same atomic number but a different mass). Examples that may be mentioned, in a non-limiting manner, include isotopes of the hydrogen atom, tritium and deuterium, and also those of carbon, C-13 and C-14.

According to the invention, the term “alkyl” denotes a linear or branched hydrocarbon-based radical advantageously containing from 1 to 6 carbon atoms, such as methyl, ethyl, propyl, isopropyl, butyl, isobutyl, tert-butyl, pentyl, neopentyl, n-hexyl. C₁-C₄ groups are preferred. The alkyl groups may be substituted with an aryl group as defined below, in which case they are referred to as arylalkyl groups. Examples of arylalkyl groups are especially benzyl and phenethyl.

The term “cycloalkyl” denotes a cyclic hydrocarbon-based system, which may advantageously contain from 3 to 6 carbon atoms and may be monocyclic or polycyclic. Cyclopropyl and cyclohexyl groups may especially be mentioned.

The “alkenyl” groups are linear, branched or cyclic hydrocarbon-based radicals comprising one or more double bonds. They advantageously contain from 2 to 6 carbon atoms and, preferentially, one or two double bonds. The alkenyl groups may be substituted with an aryl group as defined below, in which case they are referred to as arylalkenyl groups.

“Alkynyl” groups are linear or branched hydrocarbon-based radicals comprising one or more triple bonds. They advantageously contain from 2 to 6 carbon atoms and, preferentially, one or two triple bonds. The alkynyl groups may be substituted with an aryl group as defined below, in which case they are referred to as arylalkynyl groups.

The “alkoxy” groups correspond to the alkyl and cycloalkyl groups defined above linked to the nucleus via an —O— bond (ether). Mention may be made most particularly of methoxy, ethoxy, n-propyloxy, i-propyloxy, n-butoxy, s-butoxy, t-butoxy, n-pentoxy, and s-pentoxy groups.

The “acyl” groups correspond to the alkyl, cycloalkyl and aryl groups defined above, linked to the nucleus via a —CO bond. Examples of acyl groups that may especially be mentioned include acetyl, propionyl, cyclohexylcarbonyl and benzoyl groups.

The “aryl” groups are mono-, bi- or tricyclic aromatic hydrocarbon-based systems, preferentially monocyclic or bicyclic aromatic hydrocarbon-based systems containing from 6 to 18 carbon atoms and even more preferentially 6 carbon atoms. Examples that may be mentioned include phenyl, naphthyl and biphenyl groups.

The “heteroaryl” groups denote aromatic hydrocarbon-based systems defined above comprising one or more ring heteroatoms. They are preferentially cyclic aromatic hydrocarbon-based systems containing from 5 to 18 carbon atoms and one or more ring heteroatoms, especially from 1 to 4 ring heteroatoms chosen from N, O and S. Among the preferred heteroaryl groups that may especially be mentioned are benzothienyl, benzofuryl, pyrrolidinyl, thiazolyl, thienyl, furyl, pyranyl, pyrrolyl, 2H-pyrrolyl, imidazolyl, benzimidazolyl, pyrazolyl, isothiazolyl, isoxazolyl and indolyl groups, this list not being limiting.

The aryl and heteroaryl groups may be substituted with an alkyl, alkenyl or alkynyl group as defined above. In the case of an aryl or heteroaryl substituted with an alkyl group, it is referred to as an alkylaryl group. Examples of alkylaryl groups are essentially tolyl, mesithyl and xylyl. In the case of an aryl or heteroaryl substituted with an alkenyl group, it is referred to as an alkenylaryl group. An example of an alkenylaryl group is especially the cinnamyl group. In the case of an aryl or heteroaryl substituted with an alkynyl group, it is referred to as an alkynylaryl group.

The “heterocycles” denote aromatic or non-aromatic hydrocarbon-based systems comprising one or more ring heteroatoms. They are preferably cyclic hydrocarbon-based systems containing from 5 to 18 carbon atoms and one or more ring heteroatoms, especially from 1 to 4 ring heteroatoms chosen from N, O and S. Among the preferred heterocycles that may especially be mentioned are morpholine, piperazine, piperidine, tetrahydrofuran, oxazolidine, and isoxazoline, this list not being limiting.

The term “halogen” means a fluorine, chlorine, bromine or iodine atom.

The term “heteroatom” means an atom chosen from O, N and S.

Artofisopam or dextofisopam is one of the two purified enantiomers of tofisopam. It is more specifically the compound of formula (5R)-1-(3,4-dimethoxyphenyl)-5-ethyl-7,8-dimethoxy-4-methyl-5H-2,3-benzodiazepine. Specifically, in order to define the chirality it is necessary to know both the optical rotation defined by the dextrorotatory (+) or levorotatory (−) terms and the absolute configuration of the molecule defined by the terms R (rectus) or S (synister). As a result, the artofisopam-based compounds claimed in this patent application, and also the totally or partially purified (5S) isomers thereof, may take, depending on the substituent R₅, the R or S absolute configuration.

In one particular embodiment, the invention relates to the compounds of formula I or II, in which:
R₁ and R₃, independently of each other, are chosen from a hydrogen atom, a (C₁-C₆) alkyl, (C₃-C₆) cycloalkyl, alkoxyalkyl, (C₆-C₁₈) aryl or alkoxy(aryl) group, or a group OR₂, independently of each other, are chosen from a hydrogen atom and a (C₁-C₆) alkyl or (C₆-C₁₂) aryl group.
   R₄ is chosen from a halogen atom, a (C₁-C₇) alkyl, (C₂-C₇) alkenyl, (C₂-C₇) alkynyl or phenyl group or a group (C═O)R₂, OR₂, SR₂ or NR₂R_{3′} in which R₂ and R₃′ are as defined above,
   R₅ is chosen from (C₁-C₆) alkyl, (C₂-C₆) alkenyl, (C₃-C₆) cycloalkyl and (C₂-C₆) alkynyl groups,
R₇ and R₈, independently of each other, are chosen from a hydrogen atom, a (C₁-C₆) alkyl group or a group OR₂, SR₂ or NR₂R_{3′} in which R₂ and R_{3′} are as defined above,

the alkyl, cycloalkyl, aryl, heteroaryl, alkenyl and alkynyl groups and the hydrocarbon-based chain defined above being optionally substituted with one or more substituents, which may be identical or different, preferably chosen from a halogen atom, an OH, ═O, NO₂, NH₂, CN, COOH or CF₃ group, a (C₁-C₆) alkoxy group and a group NHCOR₂ or CONR₂R_{3′}, in which R₂ and R₃ are as defined above, the salts thereof, and the optically pure isomers.

In another embodiment, the invention relates the following compounds:
(5R)-1-(3,4-dimethoxyphenyl)-5-ethyl-7,8-diethoxy-4-methyl-5H-2,3-benzodiazepine,
(5S)-1-(3,4-dimethoxyphenyl)-5-ethyl-7,8-diethoxy-4-methyl-5H-2,3-benzodiazepine,
(5R)-1-(3,4-dimethoxyphenyl)-5-ethyl-7,8-dimethoxy-4-methyl-5H-2,3-benzodiazepine,
(5S)-1-(3,4-dimethoxyphenyl)-5-ethyl-7,8-dimethoxy-4-methyl-5H-2,3-benzodiazepine,
(5R)-1-(2-methoxyphenyl)-5-ethyl-7,8-dimethoxy-4-methyl-5H-2,3-benzodiazepine,
(5S)-1-(2-methoxyphenyl)-5-ethyl-7,8-dimethoxy-4-methyl-5H-2,3-benzodiazepine,
(5R)-1-(2-methoxyphenyl)-5-ethyl-7,8-diethoxy-4-methyl-5H-2,3-benzodiazepine,
(5S)-1-(2-methoxyphenyl)-5-ethyl-7,8-diethoxy-4-methyl-5H-2,3-benzodiazepine,
(5R)-1-(2-hydroxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5R)-1-(3,4-dimethoxyphenyl)-5-ethyl-7,8-diethoxy-3,5-dihydro-4H-2,3-benzodiazepin-4-one,
(5S)-1-(3,4-dimethoxyphenyl)-5-ethyl-7,8-diethoxy-3,5-dihydro-4H-2,3-benzodiazepin-4-one,
(5R)-1-(3,4-dimethoxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dimethoxy-3,5-dihydro-4H-2,3-benzodiazepin-4-one,
(5R)-1-(2-methoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5R)-1-(2-hydroxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5R)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5R)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-hydroxy-8-methoxy-4-methyl-5H-2,3-benzodiazepine,
(5R)-1-(3,4-dimethoxyphenyl)-5-ethyl-7-methoxy-8-hydroxy-4-methyl-5H-2,3-benzodiazepine,
(5S)-1-(3,4-dimethoxyphenyl)-5-ethyl-7-methoxy-8-hydroxy-4-methyl-5H-2,3-benzodiazepine,
(5R)-1-(3,4-dimethoxyphenyl)-5-ethyl-7,8-dihydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5R)-1-(3,4-diethoxyphenyl)-5-ethyl-7,8-dihydroxy-4-(prop-1-ynyl)-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5R)-1(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-ethynyl-5H-2,3-benzodiazepine,
(5R)-1-(3,4-diethoxyphenyl)-5-ethyl-7,8-dihydroxy-4-acetyl-5H-2,3-benzodiazepine,
(5S)-1-(3,4-diethoxyphenyl)-5-ethyl-7,8-dihydroxy-4-acetyl-5H-2,3-benzodiazepine,
   and also the salts thereof.

The invention also relates to the use of compounds compounds characterized in that they are chosen from the group consisting of the following compounds, alone or as mixtures:
(5R)-1(3,4-dimethoxyphenyl)-5-ethyl-7,8-diethoxy-4-methyl-5H-2,3-benzodiazepine,
(5S)-1(3,4-dimethoxyphenyl)-5-ethyl-7,8-diethoxy-4-methyl-5H-2,3-benzodiazepine,
(5R)-1(3,4-dimethoxyphenyl)-5-ethyl-7,8-dimethoxy-4-methyl-5H-2,3-benzodiazepine,
(5S)-1-(3,4-dimethoxyphenyl)-5-ethyl-7,8-dimethoxy-4-methyl-5H-2,3-benzodiazepine,
(5R)-1-(2-methoxyphenyl)-5-ethyl-7,8-dimethoxy-4-methyl-5H-2,3-benzodiazepine,
(5S) -1-(2-methoxyphenyl)-5-ethyl-7,8-dimethoxy-4-methyl-5H-2,3-benzodiazepine,
(5R)-1-(2-methoxyphenyl)-5-ethyl-7,8-diethoxy-4-methyl-5H-2,3-benzodiazepine,
(5S)-1-(2-methoxyphenyl)-5-ethyl-7,8-diethoxy-4-methyl-5H-2,3-benzodiazepine,
(5R)-1-(2-hydroxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5R)-1-(3,4-dimethoxyphenyl)-5-ethyl-7,8-diethoxy-3,5-dihydro-4H-2,3-benzodiazepin-4-one,
(5S)-1-(3,4-dimethoxyphenyl)-5-ethyl-7,8-diethoxy-3,5-dihydro-4H-2,3-benzodiazepin-4-one,
(5R)-1-(3,4-dimethoxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dimethoxy-3,5-dihydro-4H-2,3-benzodiazepin-4-one,
(5R)-1-(2-methoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5R)-1-(2-hydroxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5R)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5R)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-hydroxy-8-methoxy-4-methyl-5H-2,3-benzodiazepine,
(5R)-1-(3,4-dimethoxyphenyl)-5-ethyl-7-methoxy-8-hydroxy-4-methyl-5H-2,3-benzodiazepine,
(5S)-1(3,4-dimethoxyphenyl)-5-ethyl-7-methoxy-8-hydroxy-4-methyl-5H-2,3-benzodiazepine,
(5R)-1-(3,4-dimethoxyphenyl)-5-ethyl-7,8-dihydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5R)-1-(3,4-diethoxyphenyl)-5-ethyl-7,8-dihydroxy-4-(prop-1-ynyl)-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5R)-1-(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-ethynyl-5H-2,3-benzodiazepine,
(5R)-1-(3,4-diethoxyphenyl)-5-ethyl-7,8-dihydroxy-4-acetyl-5H-2,3-benzodiazepine,
(5S)-1-(3,4-diethoxyphenyl)-5-ethyl-7,8-dihydroxy-4-acetyl-5H-2,3-benzodiazepine, and also the salts thereof, for the preparation of a medicament for inhibiting type 2 and 4 phosphodiesterases.

The invention also relates to the use of these compounds for the preparation of a medicament for preventing or treating osteoporosis in a mammal.

The invention also relates to the use of these compounds for the preparation of a medicament for preventing or treating psoriasis in a mammal.

The invention also relates to the use of these compounds for the preparation of a medicament for treating bone resorption of the oral gum bones.

The invention also relates to the use of these compounds for the preparation of a medicament for treating ocular diseases, for instance macular degeneration and retinopathies.

The modes of preparation for obtaining racemic Tofisopam or derivatives thereof are described in the literature.

The novel compounds according to the invention are obtained via the following synthetic routes:

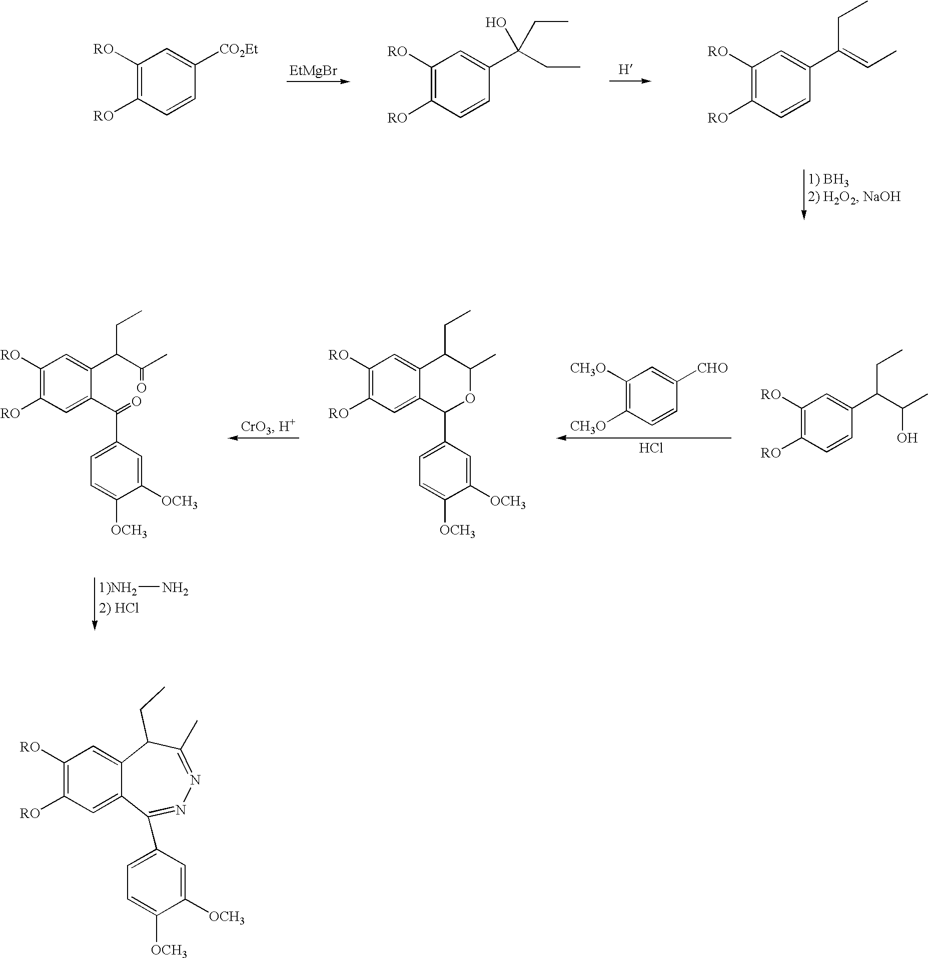

After a synthesis as described above, the isomers are separated, via known techniques suited to the particular nature of benzodiazepine and especially to the existence of four conformers.

In another embodiment, the invention also relates to a process for the stereospecific synthesis of the compounds according to the invention, which comprises the following steps: 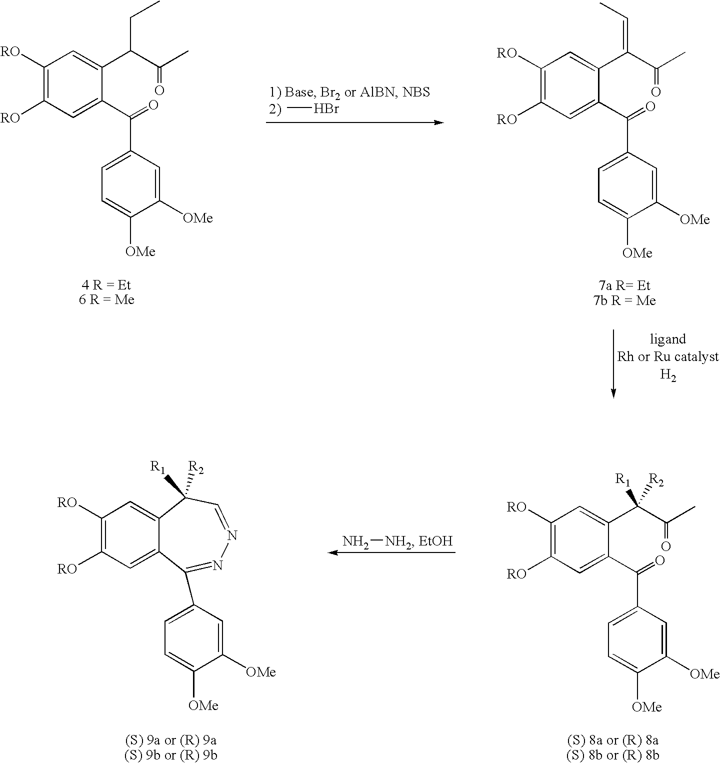
the alkenes 7 are synthesized in quantitative yields from compounds 4 and 6 (Muller, A. et al. J. Org. Chem. 1954, 19, 1533-1541) by treatment with a base and addition of bromine (or radical bromination) followed by the elimination of HBr;
asymmetric catalytic hydrogenation of the compounds 7 is performed in the presence of (R) or (S) BINAP or BINOP derivatives and of Ro or Rh catalysts under an H₂ atmosphere to obtain the enantiomers of 8 (R or S);
these enantiomers 8 are then cyclized by treatment with hydride to give the (R) or (S) benzodiazepine 9.

The compounds according to the invention are especially capable of increasing the synthesis and/or release of neurotrophic factors and of having a neuroprotective effect.

Among the growth factors induced by the administration of these novel derivatives, mention may be made especially, in a non-limiting manner, of: NGF (Nerve Growth Factor), NT-3, BDNF (Brain-Derived Neurotrophic Factor), Ciliary Neurotrophic Factor (CNTF), bFGF (basic Fibroblast Growth Factor), neurotrophin-3, protein S-100 beta (Rathbone, M. P. et al. Prog. Neurobiol. (1999), 59, 663-690), and also other neurotrophic factors involved in the survival and regeneration of sensitive or motor neurons. This increase in the synthesis and/or release of neutrotrophic factor(s) is the consequence of a modulation of carbon monoxide-dependent guanylate cyclase and/or of inhibition of a phosphodiesterase. In both cases, an increase in the intracellular levels of cGMP will be observed.

The compounds according to the invention may act on one or the other enzyme (guanylate cyclase or phosphodiesterase) or combine a simultaneous action on these two targets. In the latter case, synergistic action will be obtained and will be reflected by a strong intracellular increase in cGMP, possibly combined with an increase in cAMP. For certain conditions or pathologies, a mixed phosphodiesterase inhibitor, i.e. an inhibitor that acts simultaneously on at least two different phosphodiesterase families (especially PDE2 and PDE4) will be preferred. For example, a type 4 phosphodiesterase (PDE4) inhibitor will make it possible to treat the inflammatory component relating to the target conditions or pathologies. This anti-inflammatory effect is especially the consequence of a strong dose-dependent decrease in the production of type alpha tumour necrosis factor (TNF-α) by the pro-inflammatory cells. Moreover, a PDE4 inhibitor will also make it possible to treat depression, dementia or anxiety.

Certain molecules according to the invention are powerful and selective inhibitors of type 4 phosphodiesterase (PDE4), which may or may not act simultaneously on increasing the synthesis and the release of one or more neurotrophic factors. These PDE4 inhibitors have demonstrated a pronounced anti-inflammatory effect that may advantageously be used for treating and preventing inflammatory and autoimmune diseases. PDE4 inhibitors (IPDE4) are particularly advantageous for treating asthma and chronic obstructive bronchopathies, but also other conditions, for instance rhinitis, acute respiratory distress syndrome, allergies, dermatitis, psoriasis, rheumatoid arthritis, multiple sclerosis, dyskinesia, glomerulonephritis, osteoarthritis, cancer, septic shock, AIDS, Crohn's disease, osteoporosis, or rheumatoid arthritis, or obesity. IPDE4s also have central effects that are particularly advantageous for treating depression, anxiety, schizophrenia, bipolar disorder, attention deficits, fibromyalgia, Parkinson's and Alzheimer's diseases, amyotrophic sclerosis, multiple sclerosis, Lewy body dementia and other psychiatric disorders. The novel PDE4 inhibitors are advantageously free of emetic and hypotensive effects.

Certain compounds of the invention advantageously have anti-inflammatory effects, immunomodulatory, neurological, antimicrobial or antiviral properties or cardiovascular effects. These properties combined with the main activity may be due to a pharmacophore other than that which generates the main property. The combination of these two properties in the same molecule is particularly advantageous for the treatment of Alzheimer's disease, Parkinson's disease, AIDS and diabetes, and also memory disorders, especially those associated with senescence. In certain cases, an inhibitory property on PDE, on cycline-dependent kinases, on monoaminooxygenase or on the ‘multidrug’ transporter will allow these combined properties to be obtained.

The compounds according to the invention also advantageously have excellent central tropism and are advantageously free of hyperalgic and pro-inflammatory effects. Other compounds are advantageously free of central effects and penetrate very little into the central nervous system.

The compounds of the invention may be prepared from commercial products, using a combination of chemical reactions known to those skilled in the art.

Thus, the pharmaceutical compositions containing the compounds according to the invention may be used in the treatment of neurodegenerative or neurological disorders of the central and peripheral systems, including age-related cognitive disorders such as senility and Alzheimer's disease, nerve lesions, prion diseases (especially spongiform encephalopathies such as Creutzfeldt-Jakob disease), peripheral neuropathies, including neuropathies associated with the administration of medicaments (oncolytic, etc.), Down's syndrome, strokes and conditions involving spasms such as epilepsy. The compounds according to the invention are particularly advantageous in the treatment of pathologies or conditions in which the central or peripheral neuronal functions are impaired, and more particularly in conditions or disorders resulting from excessive neuronal death, for instance neurodegenerative or neurological disorders of the central and peripheral systems of chronic or acute nature. Mention may be made especially, in a non-limiting manner, of age-related cognitive and mental disorders (especially senility), Alzheimer's disease, Parkinson's disease, amyotrophic lateral sclerosis, Down's syndrome, multiple sclerosis, Huntington's disease, strokes, peripheral neuropathies (including neuropathies associated with the taking of medicaments or with diabetes), retinopathies (especially pigmentary retinitis), traumas (spinal column accidents, compression of the optic nerve following glaucoma and, in general, any central or peripheral nerve lesion, etc.) or neuronal disorders caused by the action of chemical products, and also disorders associated with these conditions or diseases that may be disorders secondary to the primary pathology. In many cited cases, it is usually the progressive death of motor neurons and/or sensitive neurons that is the cause of the disorders observed. In certain cases, the pharmaceutical compositions containing the compounds according to the invention may be free of neurotrophic effects, but may act strongly as PDE2 or PDE4 inhibitors or may combine a simultaneous action on these two enzymes (mixed PDE2/PDE4 inhibitor). These compounds are particularly advantageous for treating inflammatory and autoimmune diseases.

This treatment may also be administered preventively, to patients at risk of developing these same diseases.

Certain compounds of the invention have anti-inflammatory effects, immunomodulatory, neurological, antimicrobial or antiviral properties or cardiovascular effects. The combination of these two properties in the same molecule is particularly advantageous for the treatment of Alzheimer's disease, Parkinson's disease and AIDS, and also memory disorders, especially those associated with senescence.

The compounds of the invention are also particularly advantageous for treating pathologies of the central nervous system, more specifically pathologies such as depression, schizophrenia, bipolar disorder, attention deficit disorders, conditions involving spasms such as epilepsy, fibromyalgia and Lewy body dementia.

For the purposes of the invention, the term “treatment” denotes a treatment that is both preventive and curative, which may be used alone or in combination with other agents or treatments. In addition, it may be a treatment for chronic or acute disorders.

The invention relates to the following compounds, salts thereof and the R or S isomers, which may be separated:

1-(3,4-dimethoxyphenyl)-4-methyl-5-ethyl-7,8-diethoxy-5H-2,3-benzodiazepine,

1-(3,4-dimethoxyphenyl)-4-ethynyl-5-ethyl-7,8-diethoxy-5H-2,3-benzodiazepine,

4-acetyl-1-(3,4-dimethoxyphenyl)-5-ethyl-7,8-diethoxy-5H-2,3-benzodiazepine,

4-acetyl-1-(3,4-dimethoxyphenyl)-5-ethyl-7,8-dimethoxy-5H-2,3-benzodiazepine.

The compounds according to the invention and more particularly the S isomers of the compounds according to the invention and more particularly the following compounds:
(5S)-1-(3,4-dimethoxyphenyl)-5-ethyl-7,8-diethoxy-4-methyl-5H-2,3-benzodiazepine,
(5S) -1-(3,4-dimethoxyphenyl) -5-ethyl-7, 8-dimethoxy-4-methyl-5H-2,3-benzodiazepine,
(5S) -1-(2-methoxyphenyl) -5-ethyl-7, 8-dimethoxy-4-methyl-5H-2,3-benzodiazepine,
(5S)-1-(2-methoxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5S)-1-(3,4-dimethoxyphenyl)-5-ethyl-7,8-diethoxy-3,5-dihydro-4H-2,3-benzodiazepin-4-one,
(5S)-1-(3,4-dimethoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-methoxy-8-hydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5S)-1-(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-acetyl-5H-2,3-benzodiazepine,

and also the salts thereof, increase the synthesis and/or release of neurotrophic factors and have a neuroprotective effect.

The invention relates to the use of S-tofisopam, or (5S)-1-(3,4-dimethoxyphenyl)-5-ethyl-7,8-dimethoxy-4-methyl-5H-2,3-benzodiazepine, for the preparation of a medicament with a neuroprotective effect.

The compounds according to the invention may thus have local and/or systemic action, for example in the case of ocular diseases, for instance macular degeneration, retinopathies and other pathologies involving degeneration of the cells of the eye, for which these compounds are claimed. The presentation in the galenical form of eye drops or of injectable forms may be indicated for such pathologies. A preparation containing between 0.1% and 5% of active principle such as S-tofisopam or derivatives is an example of a formulation for treating these ocular pathologies.

The invention also relates to the use of the compounds according to the invention mentioned above for the preparation of a medicament for treating ocular diseases, for instance macular degeneration and retinopathies.

The invention also relates to the use of S-tofisopam, or (5S)-1-(3,4-dimethoxyphenyl)-5-ethyl-7,8-dimethoxy-4-methyl-5H-2,3-benzodiazepine, for the preparation of a medicament for treating ocular diseases, for instance macular degeneration or retinopathies.

The invention also relates to the use of S-tofisopam, or (5S)-1-(3,4-dimethoxyphenyl)-5-ethyl-7,8-dimethoxy-4-methyl-5H-2,3-benzodiazepine, for the preparation of a medicament for treating ocular diseases, for instance macular degeneration or retinopathies.

The compounds according to the invention and more particularly the S isomers of the compounds according to the invention and more particularly the following compounds:
(5S)-1(3,4-dimethoxyphenyl)-5-ethyl-7,8-diethoxy-4-methyl-5H-2,3-benzodiazepine,
(5S)-1(3,4-dimethoxyphenyl)-5-ethyl-7,8-dimethoxy-4-methyl-5H-2,3-benzodiazepine,
(5S)-1-(2-methoxyphenyl)-5-ethyl-7,8-dimethoxy-4-methyl-5H-2,3-benzodiazepine,
(5S)-1-(2-methoxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5S)-1-(3,4-dimethoxyphenyl)-5-ethyl-7,8-diethoxy-3,5-dihydro-4H-2,3-benzodiazepin-4-one,
(5S) -1-(3,4-dimethoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-methoxy-8-hydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5S) -1-(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-acetyl-5H-2,3-benzodiazepine,
   and also the salts thereof, are powerful inhibitors of type 4 phosphodiesterase, but conserve significant inhibitory activity on phosphodiesterase 2.

This activity with respect to both PDE4 and 2 is particularly advantageous in the treatment of conditions such as osteoporosis, where it makes it possible to act both on stopping bone loss and also stimulating bone regrowth.

Significant activity on the microarchitecture of the trabecular bone is especially observed, which may be due to two effects, an anabolic effect on bone formation and an inhibitory effect on bone resorption.

The compounds according to the invention and especially the following compounds:
(5S)-1-(3,4-dimethoxyphenyl)-5-ethyl-7,8-diethoxy-4-methyl-5H-2,3-benzodiazepine,
(5S)-1-(3,4-dimethoxyphenyl)-5-ethyl-7,8-dimethoxy-4-methyl-5H-2,3-benzodiazepine,
(5S)-1-(2-methoxyphenyl)-5-ethyl-7,8-dimethoxy-4-methyl-5H-2,3-benzodiazepine,
(5S)-1-(2-methoxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5S)-1-(3,4-dimethoxyphenyl)-5-ethyl-7,8-diethoxy-3,5-dihydro-4H-2,3-benzodiazepin-4-one,
(5S)-1-(3,4-dimethoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-methoxy-8-hydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5S)-1-(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-acetyl-5H-2,3-benzodiazepine,
   alone or as mixtures, and the salts thereof, are also advantageous in parodontal treatments and especially either as a local application or by systemic treatment in the treatment and prevention of bone resorption of the oral gum bones, for example in the case of parodontal disease or the insertion of implants. In general, they find a particular application in the conservation of bone density and may be used especially after any insertion of implants, for example implants of the prosthesis type for reconstruction after trauma.

The compounds thus defined are used for the preparation of a medicament for inhibiting type 2 and 4 phosphodiesterases.

They are used for the preparation of a medicament for treating or preventing osteoporosis in a mammal.

They are used for the preparation of a medicament for treating or preventing bone resorption of the oral gum bones in a mammal.

The invention also relates to the use of S-tofisopam, or (5S)-1-(3,4-dimethoxyphenyl)-5-ethyl-7,8-dimethoxy-4-methyl-5H-2,3-benzodiazepine, for the preparation of a medicament for treating or preventing bone resorption of the oral gum bones in a mammal.

The compounds according to the invention and more particularly the S isomers of the compounds according to the invention and more particularly the following compounds:
(5S)-1-(3,4-dimethoxyphenyl)-5-ethyl-7,8-diethoxy-4-methyl-5H-2,3-benzodiazepine,
(5S)-1-(3,4-dimethoxyphenyl)-5-ethyl-7,8-dimethoxy-4-methyl-5H-2,3-benzodiazepine,
(5S)-1-(2-methoxyphenyl)-5-ethyl-7,8-dimethoxy-4-methyl-5H-2,3-benzodiazepine,
(5S)-1-(2-methoxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5S)-1-(3,4-dimethoxyphenyl)-5-ethyl-7,8-diethoxy-3,5-dihydro-4H-2,3-benzodiazepin-4-one,
(5S)-1(3,4-dimethoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-methoxy-8-hydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5S)-1-(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-acetyl-5H-2,3-benzodiazepine,

and also the salts thereof, are used for the preparation of a medicament for treating or preventing psoriasis in a mammal.

The invention also relates to the use of S-tofisopam, or (5S)-1-(3,4-dimethoxyphenyl)-5-ethyl-7,8-dimethoxy-4-methyl-5H-2,3-benzodiazepine, for the preparation of a medicament for treating or preventing psoriasis in a mammal.

The compounds or compositions according to the invention may be administered in different ways and in different forms. Thus, they may be administered via the injection or oral route, for instance intravenously, intramuscularly, subcutaneously, transdermally, intra-arterially, etc., the intravenous, intramuscular, subcutaneous and oral routes being preferred. For injections, the compounds are generally conditioned in the form of liquid suspensions, which may be injected by means of syringes or perfusions, for example. In this regard, the compounds are generally dissolved in buffered isotonic physiological saline solutions, etc., which are compatible with pharmaceutical use and are known to those skilled in the art. Thus, the compositions may contain one or more agents or vehicles chosen from dispersants, solubilizers, stabilizers, preserving agents, etc. Agents or vehicles that may be used in liquid and/or injectable formulations are especially methylcellulose, hydroxymethylcellulose, carboxymethylcellulose, polysorbate 80, mannitol, gelatin, lactose, plant oils, acacia, etc.

The compounds may also be administered in the form of gels, oils, tablets, eye drops, suppositories, powders, gel capsules, wafer capsules, etc., optionally by means of galenical forms or devices that ensure sustained and/or delayed release.

The compounds according to the invention may thus have local and/or systemic action, for example in the case of psoriasis, the topical action by transdermal administration in the form, for example, of a gel, cream or patch to the skin, combined with the systemic action via the blood stream and treatment of stress or anxiety inducing psoriasis attacks. A topical preparation containing between 0.1% and 10% of active principle of S-tofisopam type or derivatives, added to 5% to 15% of urea is an example of a formulation for treating psoriasis.

It is understood that the delivery rate and/or the injected dose may be adapted by a person skilled in the art as a function of the patient, the pathology concerned, the mode of administration, etc.

Typically, the compounds are administered in doses possibly ranging between 0.1 μg and 1000 mg/kg of body weight, more generally from 0.01 to 50 mg/kg and typically between 0.1 and 50 mg/kg. In addition, repeated injections may be made, when appropriate. Moreover, for chronic treatments, delay or sustained systems may be advantageous.

The invention is illustrated by the examples and the figure that follow.

Examples 1 to 5 illustrate the synthesis and the pharmacological activity of the compounds of the invention.

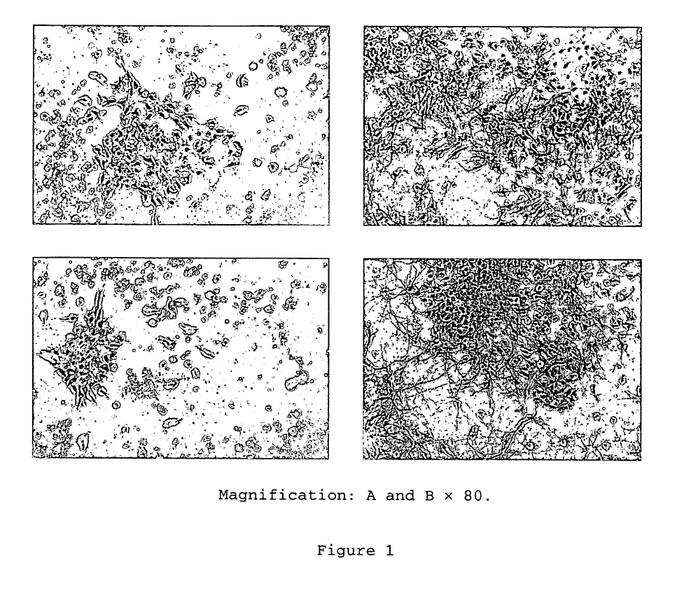

FIG. 1 shows the effect of the molecule synthesized in Example 1 on neurons in culture. The neurons are cultured in neurobasal medium using foetal rat cerebral cortex according to the procedure described in Example 1 and are photographed without staining 17 days after culturing. Culture A is a control culture without compound. The synthesized molecule was added to culture B on the eighth day after culturing, at a concentration of 50 μM.

### EXAMPLE 1

### Synthesis and Characterization of Compounds According to the Invention

The compounds below are obtained via the 6-step non-stereospecific process as described previously.

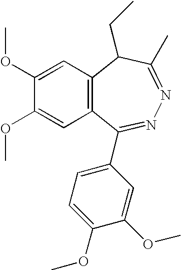

Molecular Weight=382.46

Exact Mass=382

Molecular Formula=C₂₂H₂₆N₂O₄

1-(3,4-dimethoxyphenyl)-5-ethyl-4-methyl-7,8-dimethoxy-5H-2,3-benzodiazepine (Reference: Tofisopam) ¹H RMN (250 MHz, CD₃OD) δ 1.13 (t, 3H, J=7.3 Hz, CH₃), 2.01 (s, 3H, CH₃), 2.24 (quintet, 2H, J=7.3 Hz, CH₂), 2.73 (t, 1H, J=7.3 Hz, CH), 3.71 (s, 3H, OCH₃), 3.88 (s, 3H, OCH₃), 3.90 (s, 3H, OCH₃), 3.97 (s, 3H, OCH₃), 6.86 (s, 1H, H_{Ar}), 6.95 (s, 1H, H_{Ar}), 7.03 (d, 1H, J=8.5 Hz, H_{Ar}) 7.10 (dd, 1H, J=1.8, 8.5 Hz, H_{Ar}), 7.40 (d, 1H, J=1.8 Hz, H_{Ar}). MS (IS) m/z 383 (M+1)⁺.

Via an identical procedure, the following compounds were obtained.

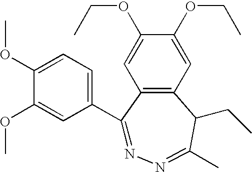

Molecular Weight=410.52

Exact Mass=410

Molecular Formula=C₂₄H₃₀N₂O₄

(S,R)-Etofisopam

1-(3,4-dimethoxyphenyl)-5-ethyl-4-methyl-7,8-diethoxy-5H-2,3-benzodiazepine ¹H RMN (250 MHz, CD₃OD) δ 1.11 (t, 3H, J=7.3 Hz, CH₃), 1.35 (t, 3H, J=7.0 Hz, CH₃), 1.57 (s, 3H, J=7.0 Hz, CH₃), 2.03 (s, 3H, CH₃), 2.26 (quintet, 2H, J=7.3 Hz, CH₂), 2.70 (t, 1H, J=7.3 Hz, CH), 3.90 (s, 3H, OCH₃), 3.92 (q, 2H, J=7.0 Hz, CH₂), 3.95 (s, 3H, OCH₃), 4.13 (q, 2H, J=7.0 Hz, CH₂), 6.90 (s, 1H, H_{Ar}), 6.97 (s, 1H, H_{Ar}), 6.99 (d, 1H, J=8.4 Hz, H_{Ar}), 7.12 (dd, 1H, J=1.8, 8.4 Hz, H_{Ar}) , 7.43 (d, 1H, J=1.8 Hz, H_{Ar}). MS (IS) m/z 411 (M+1)⁺.

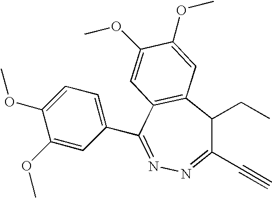

Molecular Weight=392.46

Exact Mass=392

Molecular Formula=C₂₃H₂₄N₂O₄

1-(3,4-dimethoxyphenyl)-5-ethyl-4-ethynyl-7,8-dimethoxy-5H-2,3-benzodiazepine ¹H RMN (250 MHz, CD₃OD) δ 1.11 (t, 3H, J=7.3 Hz, CH₃), 2.30 (quintet, 2H, J=7.3 Hz, CH₂), 2.80 (t, 1H, J=7.3 Hz, CH), 2.90 (s, 1H, CH), 3.70 (s, 3H, OCH₃), 3.90 (s, 3H, OCH₃), 3.91 (s, 3H, OCH₃), 4.00 (s, 3H, OCH₃), 6.88 (s, 1H, H_{Ar}), 6.91 (s, 1H, H_{Ar}), 7.08 (d, 1H, J=8.5 Hz, H_{Ar}), 7.09 (dd, 1H, J=1.8, 8.5 Hz, H_{Ar}), 7.42 (d, 1H, J=1.8 Hz, H_{Ar}). MS (IS) m/z 393 (M+1)⁺.

Its 7,8-diethoxy derivative was also synthesized: MS (IS) m/z 421 (M+1)⁺; Anal. for C₂₅H₂₈N₂O₄: Calculated: C, 71.41; H, 6.71; N, 6.66. Found: C, 71.10; H, 6.54; N, 6.60.

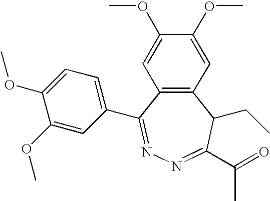

Molecular Weight=410.47

Exact Mass=410

Molecular Formula=C₂₃H₂₆N₂O₅

4-acetyl-1-(3,4-dimethoxyphenyl)-5-ethyl-7,8-dimethoxy-5H-2,3-benzodiazepine ¹H RMN (250 MHz, CD₃OD) δ 1.11 (t, 3H, J=7.3 Hz, CH₃), 2.30 (quintet, 2H, J=7.3 Hz, CH₂), 2.50 (s, 3H, CH₃), 2.80 (t, 1H, J=7.3 Hz, CH), 3.70 (s, 3H, OCH₃), 3.90 (s, 3H, OCH₃), 3.91 (s, 3H, OCH₃), 4.00 (s, 3H, OCH₃), 6.88 (s, 1H, H_{Ar}), 6.91 (s, 1H, H_{Ar}), 7.08 (d, 1H, J=8.5 Hz, H_{Ar}), 7.09 (dd, 1H, J=1.8, 8.5 Hz, H_{Ar}), 7.42 (d, 1H, J=1.8 Hz, H_{Ar}). MS (IS) m/z 411 (M+1)⁺.

Its 7,8-diethoxy derivative was also synthesized: MS (IS) m/z 439 (M+1)⁺; Anal. for C₂₅H₃₀N₂O₅: Calculated: C, 68.47; H, 6.90; N, 6.39. Found: C, 68.27; H, 6.66; N, 6.29.

The following compounds were also synthesized and optically purified:
(5S) or (5R)-1-(3,4-dimethoxyphenyl)-5-ethyl-7,8-diethoxy-4-methyl-5H-2,3-benzodiazepine,
(5S) or (5R)-1-(3,4-dimethoxyphenyl)-5-ethyl-7,8-dimethoxy-4-methyl-5H-2,3-benzodiazepine,
(5S) or (5R) -1-(2-methoxyphenyl) -5-ethyl-7,8-dimethoxy-4-methyl-5H-2,3-benzodiazepine,
(5S) or (5R)-1-(2-methoxyphenyl)-5-ethyl-7,8-diethoxy-4-methyl-5H-2,3-benzodiazepine,
(5S) or (5R) -1-(2-hydroxyphenyl) -5-ethyl-7,8-diethoxy-4-methyl-5H-2,3-benzodiazepine,
(5S) or (5R)-1-(3,4-dimethoxyphenyl)-5-ethyl-7,8-diethoxy-3,5-dihydro-4H-2,3-benzodiazepin-4-one,
(5S) or (5R)-1-(3,4-dimethoxyphenyl)-5-ethyl-7,8-dimethoxy-3,5-dihydro-4H-2,3-benzodiazepin-4-one,
(5S) or (5R)-1-(2-methoxyphenyl)-5-ethyl-7,8-dimethoxy-3,5-dihydro-4H-2,3-benzodiazepin-4-one,
(5S) or (5R)-1-(2-hydroxyphenyl)-5-ethyl-7,8-dimethoxy-3,5-dihydro-4H-2,3-benzodiazepin-4-one,
(5S) or (5R)-1-(3,4-dimethoxyphenyl)-5-ethyl-7,8-dihydroxy-3,5-dihydro-4H-2,3-benzodiazepin-4-one,
(5S) or (5R)-1-(3,4-dimethoxyphenyl)-5-ethyl-7-hydroxy-8-methoxy-4-methyl-5H-2,3-benzodiazepine,
(5S) or (5R)-1-(3,4-dimethoxyphenyl)-5-ethyl-7-methoxy-8-hydroxy-4-methyl-5H-2,3-benzodiazepine,
(5S) or (5R)-1-(3,4-dimethoxyphenyl)-5-ethyl-7,8-dihydroxy-4-methyl-5H-2,3-benzodiazepine,
(5S) or (5R)-1-(3,4-diethoxyphenyl)-5-ethyl-7,8-dihydroxy-4-(prop-1-ynyl)-5H-2,3-benzodiazepine,
(5S) or (5R)-1-(3,4-diethoxyphenyl)-5-ethyl-7,8-dihydroxy-4-ethynyl-5H-2,3-benzodiazepine,
(5S) or (5R)-1-(3,4-diethoxyphenyl)-5-ethyl-7,8-dihydroxy-4-acetyl-5H-2,3-benzodiazepine,
   and also the salts thereof.

The compounds according to the invention contain a chiral carbon atom in position 5. This atom in fact makes it possible to give rise to two different compounds that have different affinities for phosphodiesterases, especially the subtypes 2 and 4. Each of the pairs of isomers described above and also known compounds, for example tofisopam, were thus treated by enantioselective crystallization in order to obtain optically pure compounds or at least compounds with a purity of greater than 80% of one of the isomers relative to the other. Solvents such as chloroform, methanol, ethyl acetate, dichloromethane, heptane, etc. may be used in this enantioselective separation. Furthermore, the use of a chiral agent can aid the enantioselective precipitation. It is these compounds that are claimed in the present patent application and that are presented for the biological activities of the following examples. Various separation methods described in the literature may also be used, for instance the method presented in Hungarian patent 178 516.

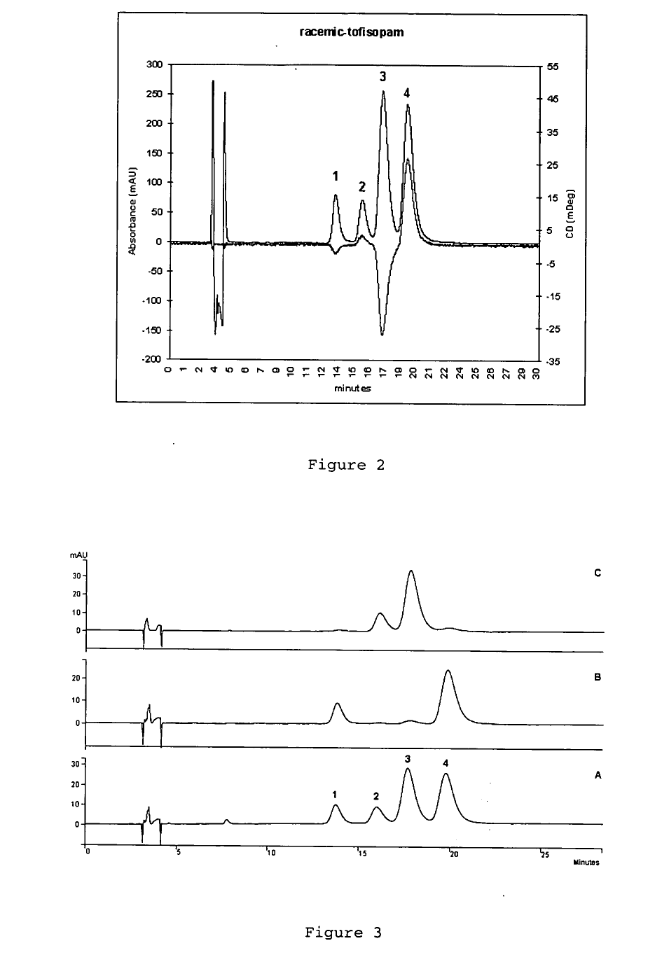

The chiral separation of the compounds was validated according to the protocol presented below: FIG. 2 shows the enantioselective separation of tofisopam under the following conditions: Chiralcel OJ-H column; Mobile phase=hexane/ethanol (90/10); flow rate=1.0 ml/minute; temperature: 30° C. The blue diagram represents the UV signal at 310 nm. The red line represents the circular dichroism diagram at 230 nm. The order of elution is R-(−)-tofisopam (1), S-(+)-tofisopam (2), S-(−)-tofisopam (3) and R-(+)-tofisopam (4).

FIG. 3 corresponds to the chromatogram obtained according to the experimental conditions: Chiralcel OJ-H at 310 nm, n-hexane/ethanol (90/10, v/v) for the chiral separation of racemic tofisopam (A), for the R-isomer after isolation (B), for the S-isomer after isolation (C). Temperature 30° C. The correspondence of the peaks is as follows: R-(−)-tofisopam (1), S-(+)-tofisopam (2), S-(−)-tofisopam (3), and R-(+)-tofisopam (4).

Detail of a procedure for the synthesis and separation of the isomers for the compound:
(5R,S)-1-(3,4-Dimethoxyphenyl)-4-methyl-5-ethyl-7,8-diethoxy-5H-2,3-benzodiazepine (5).

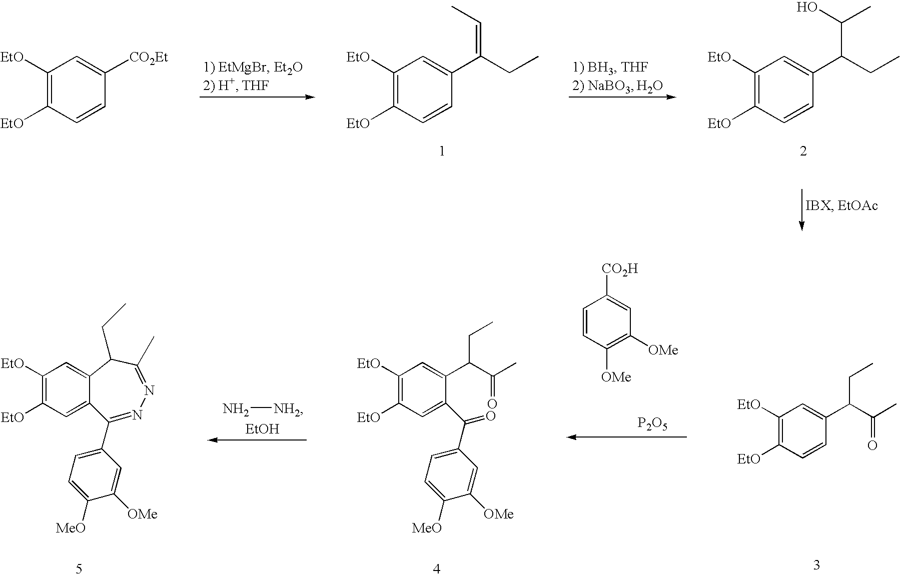

The alkene 1 was prepared quantitatively from ethyl 3,4-diethoxybenzoate by treatment with excess ethyl magnesium bromide followed by dehydration. A hydroboration-oxydation reaction on 1 gives the alcohol 2 in a yield of 94%. Oxidation of the alcohol 2 is performed in the presence of o-iodobenzoic acid (IBX) to give the ketone 3 in a yield of 98%. The acylation reaction between 3 and 3,4-dimethoxybenzoic acid leads to the diketone 4 in a yield of 32%. The final cyclization of 4 by treatment with hydrazine hydrate leads to the benzodiazepine 5 in a yield of 37%.

### 4-((1Z)-1-Ethylprop-1-enyl)-1,2-diethoxybenzene (1)

A 1.0 M solution of ethyl magnesium bromide in ether (23.5 mL, 2.5 eq) is added to a solution of ethyl 3,4-diethoxybenzoate (2.23 g, 1 eq) in ether (19 mL) at 0° C. The reaction mixture is warmed slowly to room temperature and poured into aqueous ammonium chloride solution. The organic phases are separated out and the aqueous phase is extracted with ether. The combined organic phases are dried over MgSO₄, filtered and evaporated. The residue obtained is taken up in THF (20 mL) and concentrated sulfuric acid (0.1 mL) is added. The solution is stirred at reflux for 30 minutes. The reaction mixture is poured into a sodium hydroxide solution (final pH=7-8) and ethyl acetate is added. The organic phase is separated out and the aqueous phase is extracted with ethyl acetate. The organic phases are separated out and the aqueous phase is extracted with ether. The combined organic extracts are dried over MgSO₄, filtered and evaporated. Compound 1 is obtained in a yield of 100% in the form of an oil. ¹H NMR (300 MHz, CDCl₃) δ 0.97 (t, 3H, J=7.5 Hz, CH₃), 1.44 (t, 3H, J=7.0 Hz, CH₃), 1.45 (t, 3H, J=7.0 Hz, CH₃), 1.77 (d, 3H, J=7.0 Hz, CH₃), 2.48 (q, 2H, J=7.5 Hz, CH₂), 4.08 (q, 2H, J=7.0 Hz, CH₂), 4.11 (q, 2H, J=7.0 Hz, CH₂), 5.66 (q, 1H, J=7.0 Hz, CH), 6.80-6.90 (m, 3H, H_{Ar}) ; MS (IS) m/z 235 (M⁺+1).

### 3-(3,4-Diethoxyphenyl)pentan-2-ol (2)

To a solution of 1 (1.00 g, 1 eq) in tetrahydrofuran (2 mL) is added dropwise at −5° C. a 1.0 M solution of borane-tetrahydrofuran complex in tetrahydrofuran (2.32 mL, 0.55 eq). The mixture is maintained at 0° C. for 10 minutes and then warmed to room temperature and stirred for 2 hours. H₂O (2 mL) is added slowly dropwise to the stirred solution, followed by slow addition of sodium perborate tetrahydrate (696 mg, 1.07 eq). Stirring is continued at room temperature for 2 hours. The mixture is then poured into ice-water and ethyl acetate is added. The organic phase is then separated out and the aqueous phase is extracted with ethyl acetate. The organic phases are combined and concentrated. The residue obtained is purified by preparative column chromatography (eluent: 8/2 PE/EtOAc) to give 2 in a yield of 94% in the form of an oil. ¹H NMR (300 MHz, CDCl₃) δ 0.75 (t, 3H, J=7.2 Hz, CH₃), 1.20 (d, 3H, J=6.0 Hz, CH₃), 1.44 (t, 6H, J=7.1 Hz, 2 CH₃), 1.47-1.63 (m, 1H, CH), 1.69-1.82 (m, 1H, CH₂), 2.27-2.34 (m, 1H, CH₂), 3.80-3.90 (m, 1H, CH), 4.05-4.12 (m, 4H, 2 CH₂), 6.67-6.86 (m, 3H, H_{Ar}); MS (IS) m/z 253 (M⁺+1).

### 3-(3,4-Diethoxyphenyl)pentan-2-one (3)

To a solution of compound 2 (1.10 g, 1 eq) in ethyl acetate (31 mL) is added o-iodobenzoic acid (IBX) (3.00 g, 2.5 eq) at room temperature. The suspension obtained is refluxed for 4 hours. The reaction mixture is cooled to room temperature and filtered. The filtrate is evaporated to dryness to give the pure compound 3 in a 98% yield in the form of an oil. ¹H NMR (300 MHz, CDCl₃) δ 0.85 (t, 3H, J=7.5 Hz, CH₃), 1.43 (t, 3H, J=7.0 Hz, CH₃), 1.44 (t, 3H, J=7.0 Hz, CH₃), 1.62-1.74 (m, 1H, CH₂), 1.91-2.05 (m, 1H, CH₂), 2.04 (s, 3H, CH₃), 3.41 (t, 1H, J=7.6 Hz, CH), 4.07 (q, 4H, J=7.0 Hz, CH₂), 6.69 (d, 1H, J=1.9 Hz, H_{Ar}), 6.74 (dd, 1H, J=7.9, 1.9 Hz, H_{Ar}), 6.84 (d, 1H, J=7.9 Hz, H_{Ar}); ¹³C NMR (75 MHz, CDCl₃) δ 12.0, 14.8 (2), 24.9, 28.9, 60.9, 64.5 (2), 113.2, 113.6, 120.8, 131.5, 148.0, 148.9, 208.8; MS (IS) m/z 251 (M⁺+1).

### 3-{2-[3,4-(Dimethoxyphenyl)carbonyl]-4,5-diethoxyphenyl}pentan-2-one (4)

To a solution of compound 3 (0.35 g, 1 eq) in 1,2-dichloroethane (23 mL) is added 3,4-dimethoxybenzoic acid (0.33 g, 1.28 eq). P₂O₅ (3.75 g, 9 eq) is added to the suspension. The reaction medium is stirred at room temperature for 8 hours. After addition of ice-water, the solution is neutralized by adding 1M sodium hydroxide solution (pH=7-8) and methylene chloride is added. The organic phase is separated out and the aqueous phase is extracted with methylene chloride. The combined organic phases are dried and concentrated. The crude residue is purified by preparative column chromatography (eluent: 7/3 PE/EtOAc) to afford 4 in 32% yield as a solid. M.p. 131-133° C. (EtOAc/PE) (lit: 137° C., Muller, A. et al. Monatsch. Chem. 1965, 96, 369-380); ¹H NMR (300 MHz, CDCl₃) δ 0.70 (t, 3H, J=7.5 Hz, CH₃), 1.40 (t, 3H, J=7.0 Hz, CH₃), 1.43 (t, 3H, J=7.0 Hz, CH₃), 1.55-1.68 (m, 1H, CH₂), 1.91-1.98 (m, 1H, CH₂), 2.10 (s, 3H, CH₃), 3.93 (s, 3H, CH₃), 3.93-4.00 (m, 1H, CH), 3.95 (s, 3H, CH₃), 4.00 (q, 2H, J=7.0 Hz, CH₂), 4.11 (q, 2H, J=7.0 Hz, CH₂), 6.76 (s, 1H, H_{Ar}), 6.85 (d, 1H, J=8.5 Hz, H_{Ar}), 6.85 (s, 1H, H_{Ar}), 7.29 (dd, 1H, J=8.5, 1.9 Hz, H_{Ar}), 7.53 (d, 1H, J=1.9 Hz, H_{Ar}); ¹³C NMR (75 MHz, CDCl₃) δ 12.1, 14.7 (2), 25.5, 29.7, 55.5, 56.0, 56.2, 64.5, 64.7, 109.8, 111.6, 111.9, 114.3, 126.0, 131.1, 131.6, 131.7, 146.1, 149.1, 150.7, 153.5, 196.3, 209.2; MS (IS) m/z 415 (M⁺+1).

### 1-(3,4-Dimethoxyphenyl)-4-methyl-5-ethyl-7,8-diethoxy-5H-2,3-benzodiazepine (5)

A suspension of compound 4 (100 mg, 1 eq) and hydrazine hydrate (0.03 mL, 2.6 eq) in ethanol (2 mL) is refluxed for 2 hours. After leaving the medium to cool to room temperature, it is saturated with gaseous HCl. The mixture is then concentrated to a volume of 0.5 mL, basified with concentrated ammonium hydroxide solution and extracted with methylene chloride. The combined organic phases are then dried and concentrated. The crude residue is purified by preparative column chromatography (eluent: 2/8 PE/EtOAc) to give compound 5 in a yield of 37% in the form of a solid. M.p. 91-93° C.; ¹H NMR (300 MHz, CDCl₃) δ major conformer 1.08 (t, 3H, J=7.4 Hz, CH₃), 1.37 (t, 3H, J=7.0 Hz, CH₃), 1.50 (t, 3H, J=7.0 Hz, CH₃), 1.97 (s, 3H, CH₃), 2.06-2.15 (m, 2H, CH₂), 2.76 (t, 1H, J=7.4 Hz, CH), 3.92 (s, 3H, CH₃), 3.93 (s, 3H, CH₃), 3.94 (q, 2H, J=7.0 Hz, CH₂), 4.19 (q, 2H, J=7.0 Hz, CH₂), 6.74 (s, 1H, H_{Ar}), 6.85 (d, 1H, J=8.5 Hz, H_{Ar}), 6.87 (s, 1H, H_{Ar}), 7.07 (dd, 1H, J=8.5, 1.9 Hz, H_{Ar}), 7.57 (d, 1H, J=1.9 Hz, H_{Ar}); MS (IS) m/z 411 (M⁺+1); Anal. Calcd for C₂₄H₃₀N₂O₄: C: 70.22; H, 7.37; N, 6.82. Found: C, 69.96; H: 7.43; N, 6.75.

### Chiral purification of (5R,S)-1-(3,4-Dimethoxyphenyl)-4-methyl-5-ethyl-7,8-diethoxy-5H-2,3-benzodiazepine (5) or etofisopam

The compound etofisopam may be analysed or purified by HPLC on a Chiral AGP (α₁-acid glycoprotein) column (ChromTech). The separation conditions optimized for the two enantiomers A and B and for the respective conformers thereof are:

Column: Chiral AGP (100×4 mm)

Flow rate: 0.9 mL.min⁻¹.

Detection : UV at 311 nm.

Mobile Phase: gradient elution mode

| Time (min) | % Water | %2-Prompanol |
|---|---|---|
| 0 | 95 | 5 |
| 11 | 95 | 5 |
| 21 | 85 | 15 |

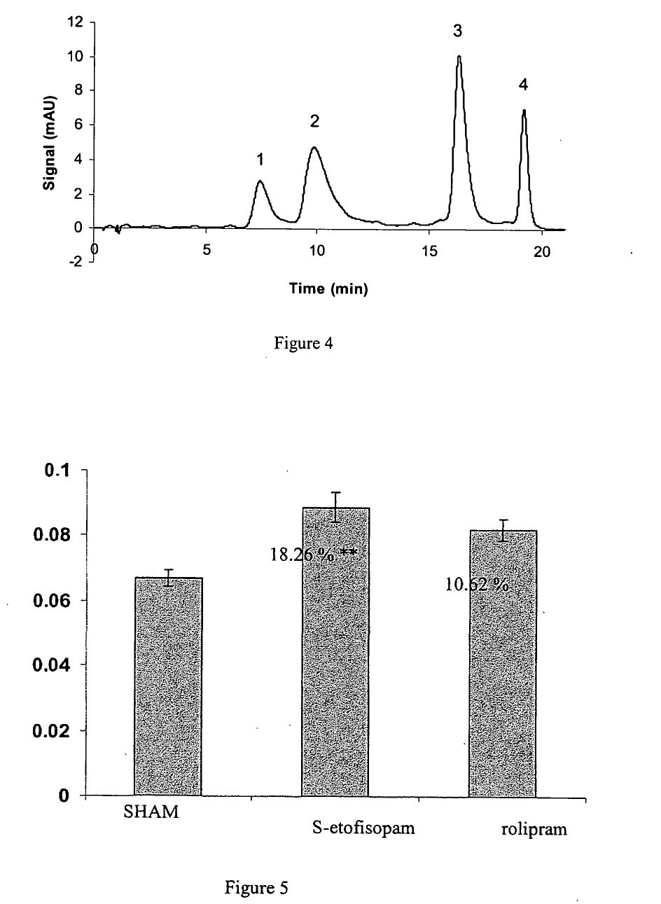

An example of a chromatogram is given in FIG. 4.

The peaks labelled 1 and 3 correspond to the two conformers of (A)-etofisopam, and the peaks labelled 2 and 4 correspond to the two conformers of (B)-etofisopam.

Using this chromatography method, peaks 1, 2, 3 and 4 were isolated by collecting the eluent at the column outlet and by producing 4 fractions labelled Fraction 1, Fraction 2, Fraction 3 and Fraction 4. Each fraction was analysed by the same chromatographic method. These analyses confirm the chemical equilibrium between the (A)-etofisopam conformers, on the one hand, and the two (B)-etofisopam conformers, on the other hand.

The purity of (A)-etofisopam is greater than 95%. The purity of (B)-etofisopam is greater than 95%.

The chiral analysis and the purification of etofisopam may be performed by simultaneously using the isocratic and gradient elution modes.

The chiral analysis and purification of etofisopam may be performed using different proportions of 2-propanol and water.

The chiral analysis and the purification of etofisopam may be performed using organic mobile phases of different nature, for instance methanol, ethanol, 1-propanol, acetonitrile, etc. in various proportions in water.

The chiral analysis and purification of etofisopam may be performed using Chiralpack OJ-H as stationary phase (Daicel) instead of AGP (ChromTech).

The chiral purification of etofisopam may be performed on the basis of the present separation using various chromatographic modes, for instance chromatography (Simulated Moving Bed, Continuous Annular Chromatography, etc.), recycling chromatography, etc.

All the methods described may be transposed to an industrial process.

The chiral purification of etofisopam may also be obtained via enantioselective crystallization. This requires the use of enantiomerically pure chiral reagents. The etofisopan racemic mixture may, for example, be converted into a diastereoisomeric mixture of the dibenzoyltartaric acid salt.

These diastereoisomers have different properties that allow them to be separated via standard methods, for instance crystallizations in suitable solvents. Such methods are described in (Toth et al., J. Heterocyclic Chem., 20:09-713 (1983)) for tofisopam.

Details of the enantioselective synthesis for the following compound:

### (5R)- or (5S)-1-(3,4-Dimethoxyphenyl)-4-methyl-5-ethyl-7,8-diethoxy-5H-2,3-benzodiazepine

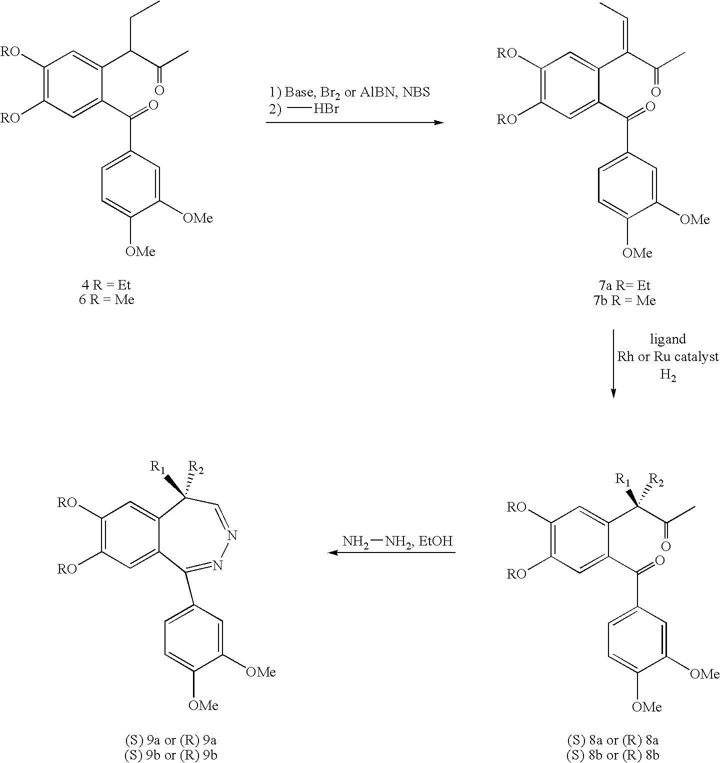

### 3-{2-[3,4-Dimethoxyphenyl)carbonyl]-4,5-diethoxyphenyl}pent-3-en-2-one (7a)

To a solution of compound 4 (100 mg, 1 eq) in THF (5 ml) is added a 1M solution of LDA in tetrahydrofuran (0.26 mL, 1.1 eq) at −78° C. The reaction mixture is stirred at −78° C. for 15 minutes, and bromine (42 mg, 1.1 eq) in terahydrofuran (5 mL) is then added. The solution is warmed to room temperature over 1 hour. The solvent is evaporated off. The residue is taken up in water and ethyl acetate. The organic phase is separated out and the aqueous phase is extracted with ethyl acetate. The combined organic phases are dried and concentrated. The crude residue is dissolved in 5 ml of THF and treated with sodium hydroxide solution. The solution is refluxed for 1 hour. The solvent is evaporated off. The residue is taken up in water and ethyl acetate. The organic phase is separated out and the aqueous phase is extracted with ethyl acetate. The combined organic phases are dried and concentrated. The crude residue is purified by preparative column chromatography (eluent: 7/3 PE/EtOAc) to give compound 7a in a yield of 65%, in the form of an oil. ¹H NMR (300 MHz, CDCl₃) δ 1.39 (t, 3H, j=7.0 Hz, CH₃), 1.42 (t, 3H, J=7.0 Hz, CH₃), 1.80 (d, 3H, J=7.0 Hz, CH₃), 2.08 (s, 3H, CH₃), 3.92 (s, 3H, CH₃), 3.94 (s, 3H, CH₃), 3.98 (q, 2H, J=7.0 Hz, CH₂), 4.10 (q, 2H, J=7.0 Hz, CH₂), 5.60 (q, 1H, J=7.0 Hz, CH), 6.74 (s, 1H, H_{Ar}), 6.80 (d, 1H, J=8.5 Hz, H_{Ar}), 6.83 (s, 1H, H_{Ar}), 7.32 (dd, 1H, J=8.5, 1.9 Hz, H_{Ar}) , 7.51 (d, 1H, J=1.9 Hz, H_{Ar}) ; MS (IS) m/z 413 (M⁺+1).

### 3-{2-[3,4-Dimethoxyphenyl)carbonyl]-4,5-diethoxyphenyl}pent-3-en-2-one (7b)

Following the same method as that described for compound 7a, compound 7b is prepared from 3-{2-[3,4-dimethoxyphenyl)carbonyl]-4,5-dimethoxyphenyl}pentan-2-one 6 in a yield of 70%.

General Method for the Asymmetric Hydrogenation of 7

A glass flask is placed in an autoclave, a vacuum is applied and the flask is flushed three times with argon. Next, under an argon atmosphere, the flask is filled with a 1 mM solution of ligand (S- or R-BINAP or BINOP derivatives) and 0.1 mM of catalyst (Rho or Ru) in anhydrous methylene chloride. The reaction mixture is stirred at room temperature for 5 minutes and substrate 7 (0.025 mmol) in toluene is added. After flushing with H₂ three times, the autoclave is placed under an H₂ pressure of 40 bar and the reaction medium is stirred at room temperature for 20 hours. The solvents are evaporated off. The crude residue is purified by preparative column chromatography (eluent: 7/3 PE/EtOAc) to give compound 8.

According to this preparation method, the following compounds are obtained in yields of greater than 90%:
(S) 3-{2-[3,4-dimethoxyphenyl)carbonyl]-4,5-diethoxyphenyl}pentan-2-one (8a),
(R) 3-{2-[3,4-dimethoxyphenyl)carbonyl]-4,5-diethoxyphenyl}pentan-2-one (8a),
(S) 3-{2-[3,4-dimethoxyphenyl)carbonyl]-4,5-dimethoxy-phenyl}pentan-2-one (8b) and
(R) 3-{2-[3,4-dimethoxyphenyl)carbonyl]-4,5-dimethoxy-phenyl}pentan-2-one (8b).
   General Method for the Cyclization of 8

A suspension of the enantiomer 8 (50 mg, 1 eq) and of hydrazine hydrate (0.02 mL, 2.6 eq) in ethanol (2 mL) is refluxed for 2 hours. After allowing the solution to cool to room temperature, it is saturated with gaseous HCl. The mixture is then concentrated to a volume of about 0.5 mL, basified with ammonium hydroxide solution and extracted with methylene chloride. The aqueous phase is extracted with methylene chloride. The combined organic phases are dried and concentrated. The crude residue is purified by preparative column chromatography (eluent: 2/8 PE/EtOAc) to give compound 9 in a good yield.

¹H NMR spectra for (S) 1-(3,4-dimethoxyphenyl)-4-methyl-5-ethyl-7,8-diethoxy-5H-2,3-benzodiazepine (9a) and (R) 1-(3,4-dimethoxy-phenyl)-4-methyl-5-ethyl-7,8-diethoxy-5H-2,3-benzodiazepine (9a), see compound 5.

¹H NMR spectra for (S) 1-(3,4-dimethoxyphenyl)-4-methyl-5-ethyl-7,8-dimethoxy-5H-2,3-benzodiazepine (9b) and (R) 1-(3,4-dimethoxy-phenyl)-4-methyl-5-ethyl-7,8-dimethoxy-5H-2,3-benzodiazepine (9b): (300 MHz, CDCl₃) δ major conformer 1.10 (t, 3H, J=7.4 Hz, CH₃), 1.99 (s, 3H, CH₃), 2.10-2.17 (m, 2H, CH₂), 2.78 (t, 1H, J=7.4 Hz, CH), 3.77 (s, 3H, CH₃), 3.93 (s, 3H, CH₃), 3.95 (s, 3H, CH₃), 3.98 (s, 3H, CH₃), 6.74 (s, 1H, H_{Ar}), 6.86 (d, 1H, J=8.5 Hz, H_{Ar}), 6.89 (s, 1H, H_{Ar}), 7.07 (dd, 1H, J=8.5, 1.9 Hz, H_{Ar}), 7.60 (d, 1H, J=1.9 Hz, H_{Ar}); MS (IS) m/z 383 (M⁺+1); Anal. Calcd for C₂₂H₂₆N₂O₄: C: 69.09; H, 6.85; N, 7.32. Found: C, 68.88; H: 6.79; N, 7.43.

### EXAMPLE 2

### Pharmacological Activity: Stimulation of the Synthesis of Neurotrophic Factors

Compounds according to the invention were evaluated for their neurotrophic properties. The idea is thus to observe the behaviour of a neuron cell culture in the absence and presence of such molecules. The molecule used in this example is S-tofisopam.

Preparation of the Neurons

Rats of Sprague Dawley strain are raised in a laboratory to adulthood, i.e. three months after birth. They are fed ad libitum in rooms at a temperature of 22±2° C. and in which the illumination cycle is 12 hours of lighting (day) and 12 hours of darkness.

The adult animals are coupled and the female rats are separated out the next day. After 16 days, a caesarean section is performed on the gestating female rats and the foetuses are placed in a Petri dish 100 mm in diameter. They are transferred to a laminar flow fume cupboard, in sterile medium. The foetuses are individually isolated and are dissected under a binocular magnifying glass in sterile medium. The cerebral cortex is isolated and placed in a tube containing antibiotic-free neurobasal medium. The tissue is dissociated by suction-ejection, into individual cells in a volume of 2 ml. The cell suspension is then delicately deposited on 2-ml of inactivated foetal calf serum. The tube is centrifuged at low gravity (800 g) for 5 minutes at room temperature. The cell pellet is recovered and the cells are resuspended in complete neurobasal medium. The cells are counted using a Mallassez haematimeter in the presence of trypan blue to determine the cell viability. Culturing is performed by placing 800 000 cells in Petri dishes 60 mm in diameter containing complete neurobasal medium, preheated and equilibrated in a CO₂ incubator. These dishes were lined beforehand with a polylysine coat the day before the manipulation. The incubator temperature is set at 37° C., the CO₂ level at 5% and the humidity is saturating. The Petri dishes containing the cells are then placed in the incubator.

About two hours after culturing, the cells that were refringent immediately after inoculation become black, which is the sign of adhesion to the bottom of the Petri dish. 24 hours after culturing, the neurites begin to grow. The growth continues for about 10 days, and signs of senescence then begin to appear. These cultures constitute primary neuron cultures.

Treatment of the Neurons

The neuron cultures as prepared above serve as controls. Five dishes are used in order to have a statistical approach.

In the other dishes, the test molecule is added at different concentrations: 0.1 μmol/l, 1 μmol/l and 10 μmol/l. In each case, the manipulation is repeated 5 times.

The neurons are examined by inverted phase-contrast microscope (Zeiss Axiovert 135) every day after inoculation.

The neurons are photographed at various magnifications using a photography camera and are compared between series.

Results

The presence of the molecule on the neurons is reflected by greater growth of the neurites than in the cells used as control. Thickening and lengthening of the neurites are observed in B compared with the control A (FIG. 1). Furthermore, the cell survival is increased in the presence of molecules. Specifically, the treated neurons remain alive almost four weeks after application, in contrast with the control in which all the neurons have disappeared after 15 days.

It is also noted that adding astrocyte culture supernatant contributes towards increasing the density of the neurites in the presence of the molecule, compared with the control. Finally, a much longer neuron lifetime is noted. S-tofisopam appears to play not only a neurotrophic role but also a neuroprotective role. A similar result had first been observed with tofisopam as a racemic mixture, but less significantly. The other derivatives described above also showed similar activities, especially the etofisopam isomers.

### EXAMPLE 3

### Inhibition of Cyclic Nucleotide Phosphodiesterases

Determination of the Inhibition of PDE4

This novel family of compounds was tested as inhibitors of human type 4 phosphodiesterase (source: U-937 cells) by following the process described by Torphy, T. J., Zhou, H. L. and Cieslinski, L. B. (J. Pharmacol. Exp. Ther., 1992, 263, 1195-1205). The concentration of substance that inhibits the enzymatic activity by 50% (IC₅₀) was determined at a substrate ([³H]cAMP+cAMP) concentration equal to 1 μM, the incubation time being 30 minutes at 30° C. A quantitative measurement of the hydrolysis product [³H]-5′-AMP was determined by scintillation. The compounds are compared with the rolipram control, which in this test has an IC₅₀ of 0.39 μM. The most powerful compounds according to the invention have an IC₅₀ of between 100 nM and 1 nM. For example, S-etofisopam has an activity of 20 nM.

Determination of the Inhibition of PDE2

This novel family of compounds was tested as inhibitor of human type 2 phosphodiesterase (source: U-937 cells) by following the process described by Torphy, T. J., Zhou, H. L. and Cieslinski, L. B. (J. Pharmacol. Exp. Ther., 1992, 263, 1195-1205). The concentration of substance that inhibits the enzymatic activity by 50% (IC₅₀) was determined at a substrate ([³H]cAMP 30 cAMPc) concentration equal to 1 μM, the incubation time being 30 minutes at 30° C. A quantitative measurement of the hydrolysis product [³H]-5′-AMP was determined by scintillation. The compounds are compared with the control EHNA, which in this test has an IC₅₀ of 2.1 μM. The most powerful compounds according to the invention have an IC₅₀ of between 5 μM and 10 nM. For example, S-tofisopam has an activity of 500 nM.

Determination of the Selectivity Towards PDE1, 3, 5 and 6

The compounds that are the most active on PDE2 and/or PDE4, for instance S-tofisopam, S-etofisopam and derivatives thereof, were tested for their selectivity towards the following cyclic nucleotide phosphodiesterases: PDE1 (bovine), PDE3 (human), PDE5 (human) and PDE6 (bovine) by following the processes described, respectively, by: (i, PDE1) Nicholson C. D., JACKMAN S. A. and WILKE R. (Brit. J. Pharmacol. 1989, 97, 889-897); (ii, PDE3 and PDE5) Weishaar, R. E., Burrows, S. D., Kobylarz, D. C., Quade, M. M. and Evans, D. B. (Biochem. Pharmacol., 1986, 35, 787-800); (iii, PDE6) Ballard, A. S., Gingell, C. J., Tang, K., Turner, L. A., PRICE, M. E. (J. Urol, 1998, 159, 2164-2171). The concentration of substance that inhibits the enzymatic activity by 50% (IC₅₀) was determined for PDE1 and PDE3 at a concentration of substrate ([³H]cAMP+cAMP) equal to 1 μM, the incubation time being 30 minutes at 30° C. In the case of PDE 5 and 6, the substrate used is ([³H]cGMP+cGMP) at a concentration of 1 μM for PDE5 and 2 μM for PDE6. A quantitative measurement of the hydrolysis products [³H]-5′-AMP and [³H]-5′-GMP was determined by scintillation. The compounds are compared with the following controls: 8-methoxy-IBMX (IC₅₀=2.9 μM) for PDE1, milrinone (IC₅₀=0.25 μM) for PDE3, dipyridamol (IC₅₀=0.5 μM) for PDE5, zaprinast (IC₅₀=0.38 μM) for PDE6.

The preferred molecules according to the invention, for instance S-tofisopam or S-etofisopam and derivatives thereof, have an excellent profile of power and selectivity towards type 4 phosphodiesterase or type 2 phosphodiesterase, insofar as these compounds inhibit the other PDEs, especially PDE3, more weakly. The selectivity coefficient is, for the most powerful compounds, greater than 100. Ideally, this coefficient is greater than 1000 or 10 000 for the most powerful compounds of the invention. In certain cases, molecules having similar activities for PDE2 and PDE4 were obtained. However, these compounds are selective towards other types of PDE (PDE1, 3, 5 and 6).

### EXAMPLE 4

### Anti-Inflammatory Properties of the Compounds of the Invention

The compounds according to the invention were evaluated for their anti-inflammatory properties on peripheral blood mononuclear cells (PBMC). More particularly, the cells were incubated for 24 hours in the presence of the test molecule, after activation with lipopolysaccharide (LPS) (1 μg/ml) according to the protocol described by Schindler, R., Mancilla, J., Endres, S., Ghorbani, R., Clark, S. C. and Dinarello, C. A. (Blood, 1990, 75, 40-47). After incubation, the concentrations of TNF-alpha were measured in the culture supernatants by the EIA method. The compounds are compared with the control dexamethasone, which in this test has an IC₅₀ of 4.6 μM. The most powerful compounds according to the invention have an IC₅₀ of less than 1 μM, i.e. they are appreciably more active than dexamethasone. Certain compounds of the invention have an IC₅₀ of between 100 nM and 1 nM in this test (S-tofisopam, S-etofisopam, R-etofisopam, etc.).

### EXAMPLE 5

### Neuroprotective Effect on Models of Induced Apoptosis

Neuroprotective Effect on a Model of Apoptosis Induced by Suppression of BDNF

This test was performed according to the protocol described by Estevez A. G. et al. (J. Neurosci. 1998, 18(3), 923-931). Briefly, when primary cultures of rat motor neuron embryonic cells are deprived of “brain-derived neurotrophic factor” (BDNF), an induction of neuronal “nitric oxide synthase” (NOS) was observed, resulting in the gradual death of the neurons by apoptosis: between 18 and 24 hours after making the biological preparation, more than 60% of the neurons die. In this model of induced apoptosis, the compound described in Example 1 (S-tofisopam and analogues) protects the neurons by more than 50% at a concentration of 1 μM.

Neuroprotective Effect on a Model of Peroxynitrite-Induced Motor Neuron Apoptosis

This test was performed by following the protocol described by Cassina P. et al. (J. Neurosci. Res. 2002 67(1):21-9). Briefly, the oxidative stress mediated by nitric oxide and its toxic metabolite, peroxynitrite, was associated with degeneration of the motor neurons, especially in amyotrophic lateral sclerosis. The astrocytes of the spinal column respond to extracellular concentrations of peroxynitrite by adopting a phenotype that is cytotoxic for the motor neurons. In this model of peroxynitrite-induced apoptosis, one of the compounds described in Example 1, S-tofisopam, or its derivative S-etofisopam, protects the neurons by more than 50% at a concentration of 1 μM.

### EXAMPLE 6

### Pharmaceutical Compositions

The compounds according to the invention demonstrated a true inhibitory effect on phosphodiesterases, especially 4 and 2, on the basis of S-tofisopam, which revealed an inhibition on PDE4 ten times higher than that of R-tofisopam. These compounds showed significant in vivo effects for pharmaceutical compositions comprising between 0.1 mg and 1500 mg.

### EXAMPLE 7

### Microarchitecture and Study of Bone Markers

The object is to test the effect of the components described above on the microarchitecture of the trabecular bone.

Materials and Methods:
C57BL6 mice treated for 4 weeks
   12 treated with S-tofisopam
   12 treated with Sham saline serum,
   12 treated with fluxetine (rolipram)—20 mg/kg per day),
   12 euthanized (controls—BASE).
S-tofisopam: 5 mg/kg per day; 5 days per week,
duration of the treatment: 4 weeks,
mode of injection: subcutaneous,
age of the mice at the start of the treatment: 12 weeks.
Standard living and feeding conditions (light, water, feed, free activity),
Study on microQCT: Skyscan 1072 microscanner—distal metaphysis of the femur (64 μm under the growth plate—voxel 6.5 μm).
   Results on the Bone Microarchitecture
No significant difference between the groups at the start of the treatment. No significant difference between the control (base) group and the Sham group.

All the significant differences are represented by an *, *p<0.05; **p<0.01
Several parameters (10) are recorded and analysed, such as:
BV/TV: represents the bone volume of the trabecular bone to the tissue volume,
Tb.N: trabecular number.

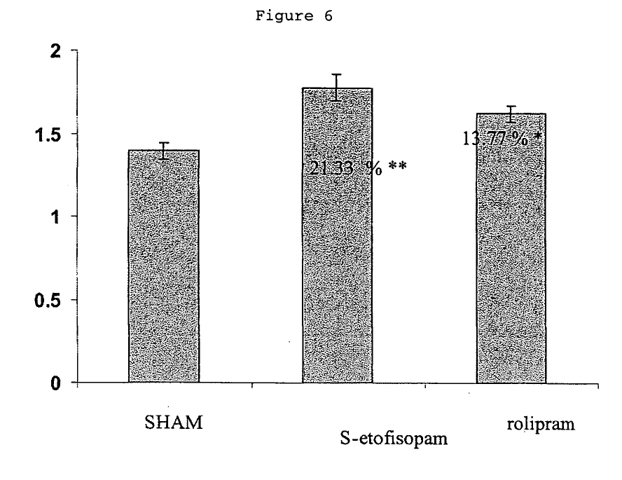

The trabecular bone volume (BV/TV, %) and the trabecular number (Tb.N) are calculated via the “Mean Intercept Length (MIL)” method. The trabecular thickness (Tb.Th) and the trabecular space (Tb.Sp) are calculated via the method described by Hildebrand & Ruegsegger (Journal of Microscopy. 185:67-75, 1997).
BV/TV: FIG. 5
Tb.N: FIG. 6
   CONCLUSION

The results obtained suggest that S-tofisopam (5 mg/kg) has a positive effect on the bone quantity and microarchitecture. The markers and the cellular activity suggest an uncoupling effect: anabolic on bone formation, inhibitory on bone resorption. Identity results were obtained with S-etofisopam (5S-(3,4-dimethoxyphenyl)-5-ethyl-4-methyl-7,8-diethoxy-5H-2,3-benzodiazepine).

1. Compounds of general formula (I) or (II) 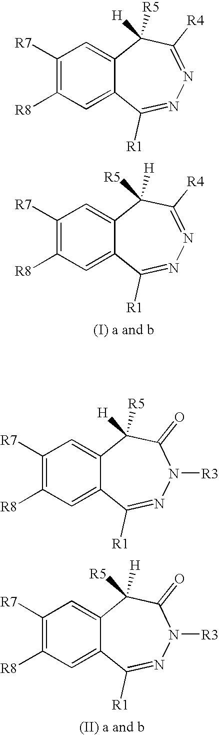
in which:
R₁ and R₃, independently of each other, are chosen from a hydrogen atom, a group (C₁-C₆) alkyl, (C₃-C₆) cycloalkyl, (C₆-C₁₈) aryl, (C₆-C₁₈)aryl(C₁-C₄)alkyl, (C₁-C₆)alkyl(C₆-C₁₈)aryl,(C₅-C₁₈) heteroaryl comprising 1 to 3 heteroatoms, or a group OR₂, SR₂ or NR₂R_{3′} in which (i) R₂ and R_{3′}, independently of each other, are chosen from a hydrogen atom, and a (C₁-C₆) alkyl, (C₃-C₆) cycloalkyl, (C₆-C₁₂) aryl, or (C₅-C₁₂) heteroaryl group comprising 1 to 3 heteroatoms or (ii) R₂ and R_{3′} together form a linear or branched hydrocarbon-based chain containing from 2 to 6 carbon atoms, optionally comprising one or more double bonds and/or interrupted with an oxygen, sulfur or nitrogen atom,
R₄ is chosen from a halogen atom, a (C₁-C₇) alkyl, (C₂-C₇) alkenyl, (C₂-C₇) alkynyl, or phenyl group or a group (C═O)R₂, OR₂, SR₂ or NR₂R_{3′} in which R₂ and R_{3′} are as defined above,
R₅ is chosen from (C₁-C₆) alkyl, (C₂-C₆) alkenyl, (C₃-C₆) cycloalkyl and (C₂-C₆) alkynyl groups,
R₇ and R₈, independently of each other, are chosen from a hydrogen atom, a (C₁-C₆) alkyl group or a Group OR₂, SR₂ or NR₂R_{3′} in which R₂and R₃ are as defined above,
the alkyl, cycloalkyl, aryl, heteroaryl, alkenyl and alkynyl groups and the hydrocarbon-based chain defined above being substituted with one or more identical or different substituents preferably chosen from a halogen atom, an OH, ═O, NO₂, NH₂, CN, COOH or CF₃ group, a (C₁-C₆) alkoxy group and a group NHCOR₂ or CONR₂R_{3′}, in which R₂ and R_{3′} are as defined above,
the salts thereof, and the pure optical isomers.

2. Compounds according to claim 1, chosen from the compounds of formula I or II, in which:
R₁ and R₃, independently of each other, are chosen from a hydrogen atom, a (C₁-C₆) alkyl, (C₃-C₆) cycloalkyl, alkoxyalkyl, (C₆-C₁₈) aryl or alkoxy(aryl) group, or a group OR₂, independently of each other, are chosen from a hydrogen atom and a (C₁-C₆) alkyl or (C₆-C₁₂) aryl group.
R₄ is chosen from a halogen atom, a (C₁-C₇) alkyl, (C₂-C₇) alkenyl, (C₂-C₇) alkynyl or phenyl group or a group (C═O)R₂, OR₂, SR₂ or NR₂R_{3′} in which R₂ and R_{3′}, are as defined above,
R₅ is chosen from (C₁-C₆) alkyl, (C₂-C₆) alkenyl, (C₃-C₆) cycloalkyl and (C₂-C₆) alkynyl groups,
R₇ and R₈, independently of each other, are chosen from a hydrogen atom, a (C₁-C₆) alkyl group or a group OR₂, SR₂ or NR₂R_{3′} in which R₂ and R_{3′}, are as defined above,
the alkyl, cycloalkyl, aryl, heteroaryl, alkenyl and alkynyl groups and the hydrocarbon-based chain defined above being optionally substituted with one or more substituents, which may be identical or different, or chosen from a halogen atom, an OH, ═O, NO₂, NH₂, CN, COOH or CF₃ group, a (C₁-C₆) alkoxy group and a group NHCOR₂ or CONR₂R₃, in which R₂ and R_{3′} are as defined above, the salts thereof, and the optically pure isomers.

3. Compounds, characterized in that they are chosen from the group consisting of the following compounds:
(5R)-1-(3,4-dimethoxyphenyl)-5-ethyl-7,8-diethoxy-4-methyl-5H-2,3-benzodiazepine,
(5S)-1-(3,4-dimethoxyphenyl)-5-ethyl-7,8-diethoxy-4-methyl-5H-2,3-benzodiazepine,
(5R)-1-(3,4-dimethoxyphenyl)-5-ethyl-7,8-dimethoxy-4-methyl-5H-2,3-benzodiazepine,
(5S)-1-(3,4-dimethoxyphenyl)-5-ethyl-7,8-dimethoxy-4-methyl-5H-2,3-benzodiazepine,
(5R)-1-(2-methoxyphenyl)-5-ethyl-7,8-dimethoxy-4-methyl-5H-2,3-benzodiazepine,
(5S)-1-(2-methoxyphenyl)-5-ethyl-7,8-dimethoxy-4-methyl-5H-2,3-benzodiazepine,
(5R)-1-(2-methoxyphenyl)-5-ethyl-7,8-diethoxy-4-methyl-5H-2,3-benzodiazepine,
(5S)-1-(2-methoxyphenyl)-5-ethyl-7,8-diethoxy-4-methyl-5H-2,3-benzodiazepine,
(5R)-1-(2-hydroxyphenyl)-5-ethyl-7,8-diethoxy-4-methyl-5H-2,3-benzodiazepine,
(5S)-1-(2-hydroxyphenyl)-5-ethyl-7,8-diethoxy-4-methyl-5H-2,3-benzodiazepine,
(5R)-1-(3,4-dimethoxyphenyl)-5-ethyl-7,8-diethoxy-3,5-dihydro-4H-2,3-benzodiazepin-4-one,
(5S)-1-(3,4-dimethoxyphenyl)-5-ethyl-7,8-diethoxy-3,5-dihydro-4H-2,3-benzodiazepin-4-one,
(5R)-1-(3,4-dimethoxyphenyl)-5-ethyl-7,8-dimethoxy-3,5-dihydro-4H-2,3-benzodiazepin-4-one,
(5S)-1-(3,4-dimethoxyphenyl)-5-ethyl-7,8-dimethoxy-3,5-dihydro-4H-2,3-benzodiazepin-4-one,
(5R)-1-(2-methoxyphenyl)-5-ethyl-7,8-dimethoxy-3,5-dihydro-4H-2,3-benzodiazepin-4-one,
(5S)-1-(2-methoxyphenyl)-5-ethyl-7,8-dimethoxy-3,5-dihydro-4H-2,3-benzodiazepin-4-one,
(5R)-1-(2-hydroxyphenyl)-5-ethyl-7,8-dimethoxy-3,5-dihydro-4H-2,3-benzodiazepin-4-one,
(5S)-1-(2-hydroxyphenyl)-5-ethyl-7,8-dimethoxy-3,5-dihydro-4H-2,3-benzodiazepin-4-one,
(5R)-1-(3,4-dimethoxyphenyl)-5-ethyl-7,8-dihydroxy-3,5-dihydro-4H-2,3-benzodiazepin-4-one,
(5S)-1-(3,4-dimethoxyphenyl)-5-ethyl-7,8-dihydroxy-3,5-dihydro-4H-2,3-benzodiazepin-4-one,
(5R)-1-(3,4-dimethoxyphenyl)-5-ethyl-7-hydroxy-8-methoxy-4-methyl-5H-2,3-benzodiazepine,
(5S)-1-(3,4-dimethoxyphenyl)-5-ethyl-7-hydroxy-8-methoxy-4-methyl-5H-2,3-benzodiazepine,
(5R)-1-(3,4-dimethoxyphenyl)-5-ethyl-7-methoxy-8-hydroxy-4-methyl-5H-2,3-benzodiazepine,
(5S)-1-(3,4-dimethoxyphenyl)-5-ethyl-7-methoxy-8-hydroxy-4-methyl-5H-2,3-benzodiazepine,
(5R)-1-(3,4-dimethoxyphenyl)-5-ethyl-7,8-dihydroxy-4-methyl-5H-2,3-benzodiazepine,
(5S)-1-(3,4-dimethoxyphenyl)-5-ethyl-7,8-dihydroxy-4-methyl-5H-2,3-benzodiazepine,
(5R)-1-(3,4-diethoxyphenyl)-5-ethyl-7,8-dihydroxy-4-(prop-1-ynyl)-5H-2,3-benzodiazepine,
(5S)-1-(3,4-diethoxyphenyl)-5-ethyl-7,8-dihydroxy-4-(prop-1-ynyl)-5H-2,3-benzodiazepine,
(5R)-1-(3,4-diethoxyphenyl)-5-ethyl-7,8-dihydroxy-4-ethynyl-5H-2,3-benzodiazepine,
(5S)-1-(3,4-diethoxyphenyl)-5-ethyl-7,8-dihydroxy-4-ethynyl-5H-2,3-benzodiazepine,
(5R)-1-(3,4-diethoxyphenyl)-5-ethyl-7,8-dihydroxy-4-acetyl-5H-2,3-benzodiazepine,
(5S)-1-(3,4-diethoxyphenyl)-5-ethyl-7,8-dihydroxy-4-acetyl-5H-2,3-benzodiazepine,
and also the salts thereof.

4. A method for inhibiting phosphodiesterases, comprising preparing and administering a pharmaceutical composition using a compound according to claim 1.

5. A method for treating or preventing pathologies involving neuronal degeneration, aging, senility, Alzheimer's disease, Parkinson's disease, amyotrophic lateral sclerosis, multiple sclerosis, Huntington's disease, Down's syndrome, pharmacodependencies, strokes, peripheral neuropathies, reinopathies, retinitis, prion diseases, spongiform encephalopathies such encephalophathies, Creutzfeldt-Jakob disease, traumas, spinal column accidents, compression of the optic nerve following glaucoma or neuronal disorders caused by the action of chemical products and nerve lesions, comprising administering to a mammal, as a medicament an effective amount of a compound according claim 1.

6. A method for treating or preventing central or peripheral diseases in a mammal, comprising administering to the mammal an effective amount of a compound according to claim 1 as a medicament for increasing the intracellular levels of cGMP and cAMP by inhibition of phosphodiesterases 2 and 4.

7. A method for inhibiting type 2 and 4 phosphodiesterases, for treating or preventing in a mammal central or peripheral diseases chosen from inflammatory diseases, chronic obstructive bronchopathies, rhinitis, dementia, acute respiratory distress syndrome, allergies, dermatitis, psoriasis, rheumatoid arthritis, infections, autoimmune diseases, multiple sclerosis, dyskinesia, glomerulonephritis, osteoarthritis, cancer, septic shock, AIDS, Crohn's disease, osteoporosis, obesity, depression, anxiety, schizophrenia, bipolar disorder, attention deficits, fibromyalgia, Parkinson's disease, Alzheimer's disease, diabetes, amyotrophic sclerosis, Lewy body dementia, conditions involving spasms such as epilepsy, senescence-related central nervous system pathologies, memory disorders, and other psychiatric disorders, comprising preparing and administering to the mammal a medicament including an effective amount of a compound according to claim 1.

8. A method for treating or preventing central or peripheral disorders in a mammal, comprising preparing and administering medicaments using a compound according to claim 1 in combination with a compound that inhibits type 2 or 4 phosphodiesterases.

9. Pharmaceutical composition comprising at least one compound according to claim 1, combined with a pharmaceutically acceptable vehicle or excipient.

10. Pharmaceutical composition comprising at least one compound according to claim 1, combined with another pharmaceutically acceptable active principle.

11. Pharmaceutical composition comprising at least 80% of the enantiomer according to claim 1.

12. Pharmaceutical composition comprising between 1 mg and 1200 mg of at least one compound according to claim 1.

13. Pharmaceutical composition comprising at least one compound according to claim 1, administered in one or more doses via the injection, oral or topical route, in the form of gels, creams, oils, tablets, eye drops or any other pharmaceutically acceptable form.

14. Salts and/or R or S isomers of the following compounds:
1-(3,4-dimethoxyphenyl)-4-methyl-5-ethyl-7,8-diethoxy-5H-2,3-benzodiazepine,
1-(3,4-dimethoxyphenyl)-4-ethynyl-5-ethyl-7,8-diethoxy-5H-2,3-benzodiazepine,
4-acetyl-1-(3,4-dimethoxyphenyl)-5-ethyl-7,8-diethoxy-5H-2,3-benzodiazepine,
4-acetyl-1-(3,4-dimethoxyphenyl)-5-ethyl-7,8-dimethoxy-5H-2,3-benzodiazepine.

15. A method for preparing a medicament using a compound according to claim 14.

16. A method for treating and preventing osteoporosis in a mammal, comprising preparing and administering a medicament using S-tofisopam, or (5S)-1-(3,4-dimethoxyphenyl)-5-ethyl-7,8-dimethoxy-4-methyl-5H.

17-23. (canceled)

24. A method for treating or preventing bone resorption of the oral gum bones in a mammal, comprising preparing and administering a medicament using S-tofisopam, or (5S)-1-(3,4-dimethoxyphenyl)-5-ethyl-7,8-dimethoxy-4-methyl-5H-2,3-benzodiazepine.

25. A method for treating psoriasis, comprising preparing and administering a medicament using S-tofisopam, or (5S)-1-(3,4-dimethoxyphenyl)-5-ethyl-7,8-dimethoxy-4-methyl-5H-2,3-benzodiazepine.

26. A method providing a neuroprotective effect, comprising preparing and administering a medicament using S-tofisopam, or (5S)-1-(3,4-dimethoxyphenyl)-5-ethyl-7,*-dimethoxy-4-methyl-5H-2,3-benzodiazepine.

27. A method for treating ocular diseases chosen among macular degeneration and retinopathies, comprising preparing and administering a medicament using S-tofisopam, or (5S)-1-(3,4-dimethoxyphenyl)-5-ethyl-7,8-dimethoxy-4-methyl-5H-2,3-benzodiazepine.
